(19) United States
(12) Patent Application Publication
(10) Pub. No.: US 2016/0032007 A1
(43) Pub. Date: Feb. 4, 2016

(54) Human Antibody Fragments Against Chondroitin Sulfate Proteoglycan 4 (CSPG4)

(71) Applicants: Darell D. Bigner, Mebane, NC (US); Liang Qu, Durham, NC (US)

(72) Inventors: Darell D. Bigner, Mebane, NC (US); Liang Qu, Durham, NC (US)

(73) Assignee: DUKE UNIVERSITY, Durham, NC (US)

(21) Appl. No.: 14/698,111

(22) Filed: Apr. 28, 2015

Related U.S. Application Data
(60) Provisional application No. 61/985,126, filed on Apr. 28, 2014.

(51) Int. Cl. C07K 16/30; A61K 47/48

(52) U.S. Cl. CPC C07K 16/3053; A61K 47/48623; C07K 2317/622; C07K 2317/34; C07K 2317/77; C07K 2319/55; C07K 2317/92

(57) ABSTRACT

Human antibody fragments against chrondroitin sulfate proteoglycan 4 can be used to deliver cytotoxic agents to cells which express CSPG4. The agents can be diagnostic or therapeutic moieties. They may be linked by covalent or non-covalent linkages to the antibody fragments. They may be produced as a genetic fusion product or joined together synthetically, for example. When the human antibody fragments are internalized by the cells to which they bind, they can carry with them the attached agents. Thus toxic agents having intracellular targets have enhanced killing upon internalization.

This invention was made with government support under CA11898-42 awarded by the National Cancer Institute. The government has certain rights in the invention.

### TECHNICAL FIELD OF THE INVENTION

This invention is related to the area of tumor therapy. In particular, it relates to antibody constructs for tumor therapy.

### BACKGROUND OF THE INVENTION

One of the biggest challenges for immunotoxin-based cancer therapy is the limitation of tumor-targeting and internalizing human monoclonal antibodies (mAbs) or antibody fragments, which are capable of delivering recombinant toxins to the cytoplasm of cancer cells effectively and specifically. There is a continuing need in the art to develop such antibodies.

### SUMMARY OF THE INVENTION

According to one embodiment of the invention a human scFv is provided that binds to an CSPG4 extracellular domain. The extracellular domain is selected from the group consisting of: amino acids 30-640; amino acids 641-1233; amino acids 1234-1586; and amino acids 1587-2222.

These and other embodiments which will be apparent to those of skill in the art upon reading the specification provide the art with

### BRIEF DESCRIPTION OF THE DRAWINGS

FIGS. 1A-1C show yeast displayed domains of the extracellular region of Chondroitin Sulfate Proteoglycan 4 (CSPG4) verified by antiCSPG4 antibodies 9.2.27 and Mel-14.

FIG. 2 shows selection of phage display non-immunized human single chain variable phage display library against yeast displayed CSPG4 domains

FIGS. 3A, 3B1-3B2, and 3C show binding of phage display human non-immunized single chain variable fragments (scFv) antibodies library on yeast display CSPG4 domains

FIG. 4A-FIG. 4I show amino acid sequences of nine unique antibodies to extracellular regions of CSPG4 (SEQ ID NO: 1-9) and their encoding nucleic acid sequences SEQ ID NO: 24-32).

FIG. 5 shows a schematic drawing showing the major steps in construction and panning of yeast display scFv library against D2A domain of human chondroitin sulfate proteoglycan 4.

FIG. 6A-6C show strategy for PCR construction of yeast mutant library. FIG. 6A. pYD1-D2A-1H10 recombinant plasmid containing the parental scFv D2A-1H10 was used as template for mutation using an upstream primer, PYD1-ECORI-F-1H10, and a downstream primer, PYD1-NotI-R-1H10. Error-prone PCR was carried out using Diversify™ PCR Random Mutagenesis Kit (Clontech) according to manufacturer's instructions. FIG. 6B. Mutant PCR products from step A were double digested using restriction enzymes EcoRI and NotI. FIG. 6C. Digested mutant PCR products from step B were cloned into pYD1 yeast display vector and transformed into the EBY100 yeast strain.

FIG. 7 shows screening of random mutagenesis yeast display library. Flow cytometry plots showing yeast display library at different stages of screening using 10 nM Nus-D2A-CSPG4 antigen. Expression of scFv on yeast surface was detected by anti-V5 antibody conjugated with APC. Binding of scFv to Nus-D2A-CSPG4 antigen was detected by PE conjugated anti-mouse IgG1 antibody. Enriched populations were shown in pink gates, and their percentages calculated as the number of gated events in total events. Round 0 refers to the naïve yeast display library before cell panning.

FIG. 8A-FIG. 8N show amino acid (SEQ ID NO: 10-23) and DNA Sequences (SEQ ID NO: 33-46) of fourteen unique D2A-1H10 Mutant scFvs. DNA sequences of the 14 mutant D2A-1H10 scFvs selected from the yeast display library with improved affinity. Red boxes indicate mutated amino acids.

FIG. 9A-9C show alignment of amino acid sequences of the parental D2A-1H10 scFv and the fourteen D2A-1H10 mutant scFvs selected from yeast display library with improved affinity. Identical residues are marked by dots and the mutant amino acids are represented by a single letter code.

FIG. 10 shows flow cytometry analysis of 14 unique D2A-1H10 mutant yeast clones reacting to 10 nM Nus-D2A-CSPG4 Antigen. FACS histogram overlay of negative control (yeasts incubated with secondary antibodies without the antigen), parental yeast clone D2A-1H10, and 14 unique D2A-1H10 mutant yeast clones reacting to 10 nM Nus-D2A-CSPG4 antigen. The seven unique clones selected for further characterization are as follows: D2A-1H10-UC3, D2A-1H10-UC4, D2A-1H10-UC5, D2A-1H10-UC8, D2A-1H10-UC10, D2A-1H10-UC12, and D2A-1H10-UC16.

FIG. 11 shows SDS-PAGE Analysis of Purified D2A-1H10-UC8 and D2A-1H10-UC12 scFvs. The mutant scFvs were expressed as inclusion bodies, refolded, and purified using cobalt column. Four micrograms of purified D2A-1H10-UC8 and D2A-1H10-UC12 scFvs were run in a SDS-PAGE gel.

FIG. 12 show flow cytometry analysis of apparent affinity (K_{D}) of D2A-1H10-UC8 and D2A-1H10-UC12 scFvs on H350 cells. FACS binding of D2A-1H10-UC8 and D2A-1H10-UC12 scFvs at different concentrations to H350 cells was used for apparent affinity determination. Experiments were repeated at least three times and representative data are presented.

FIG. 13 shows binding of mutant scFvs to H350 cell line was specific. No binding was seen against the negative control cell line HEK293.

FIG. 14 shows Affinity (KD) of D2A-1H10-UC8 by BIAcore. Experiments were repeated at least three times and representative data are presented.

FIG. 15 shows a summary of K_{D} values for D2A-1H10-UC8 and D2A-1H10-UC12 scFvs by FACS and BIAcore.

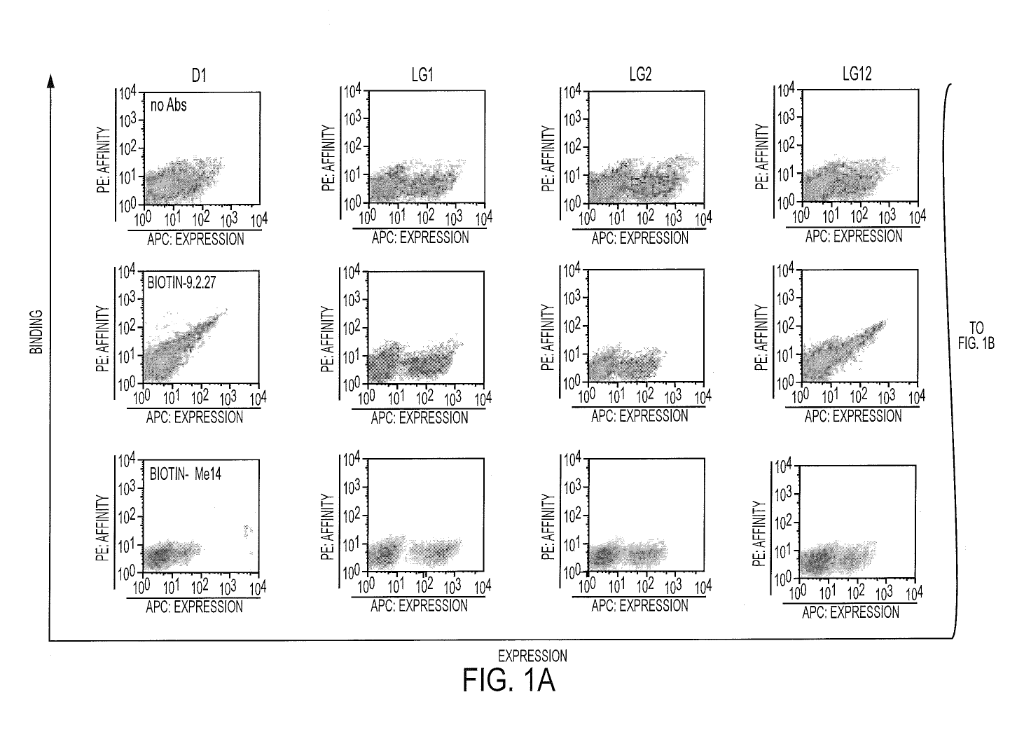

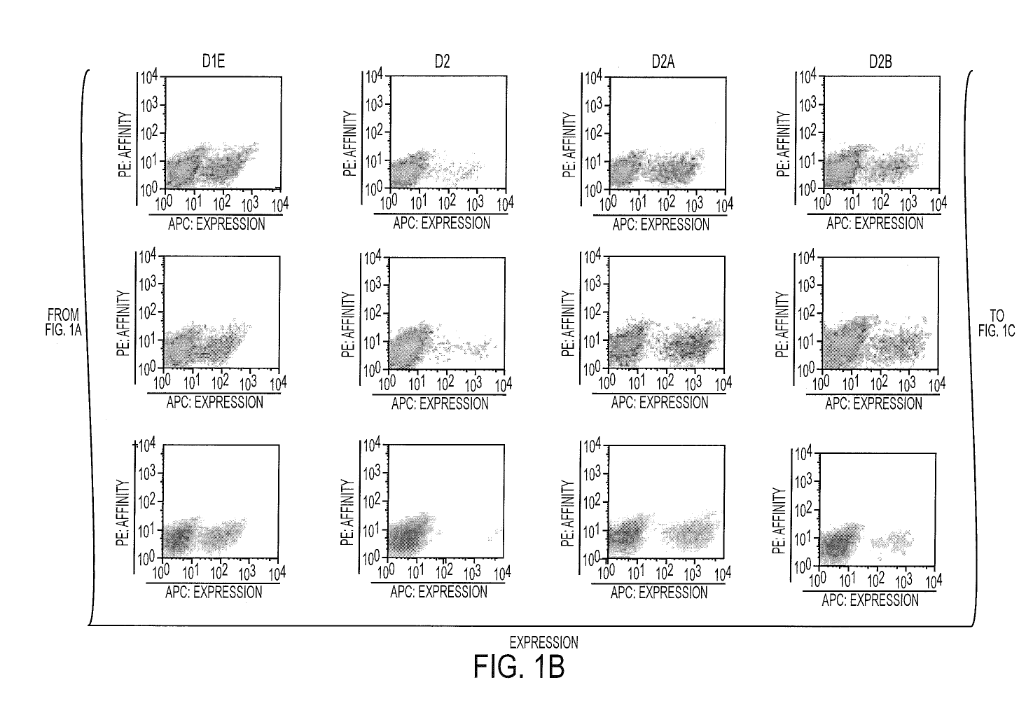

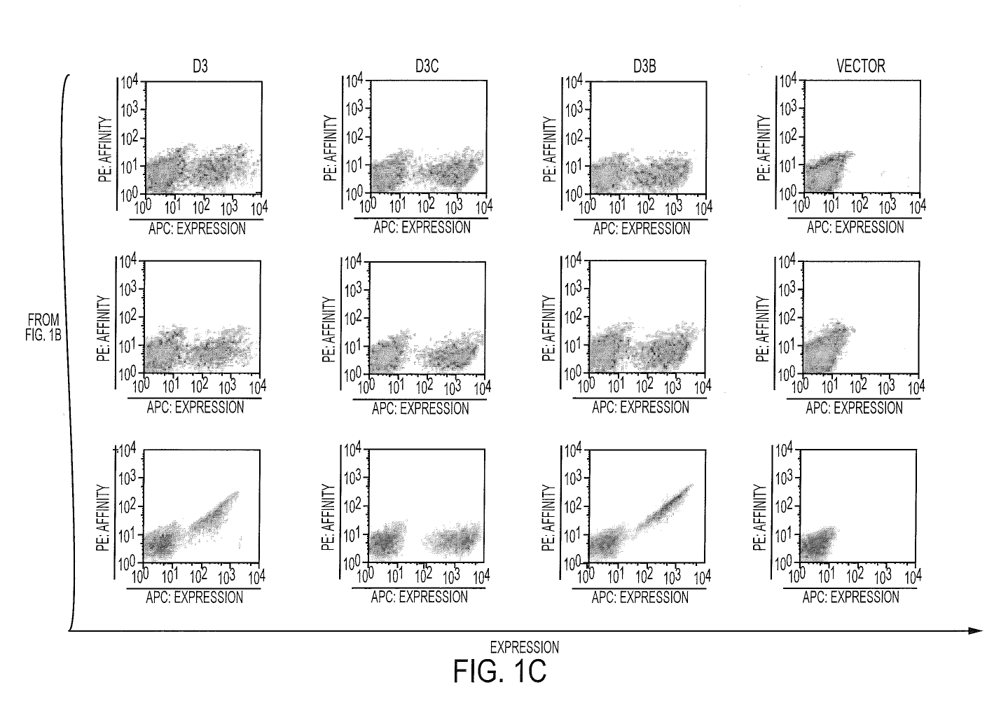

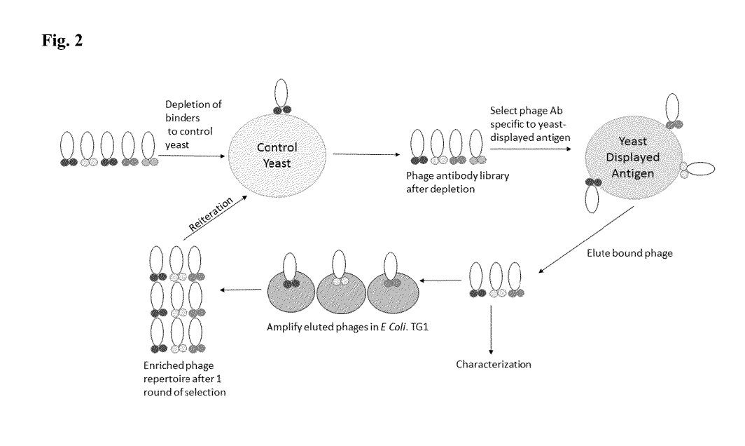

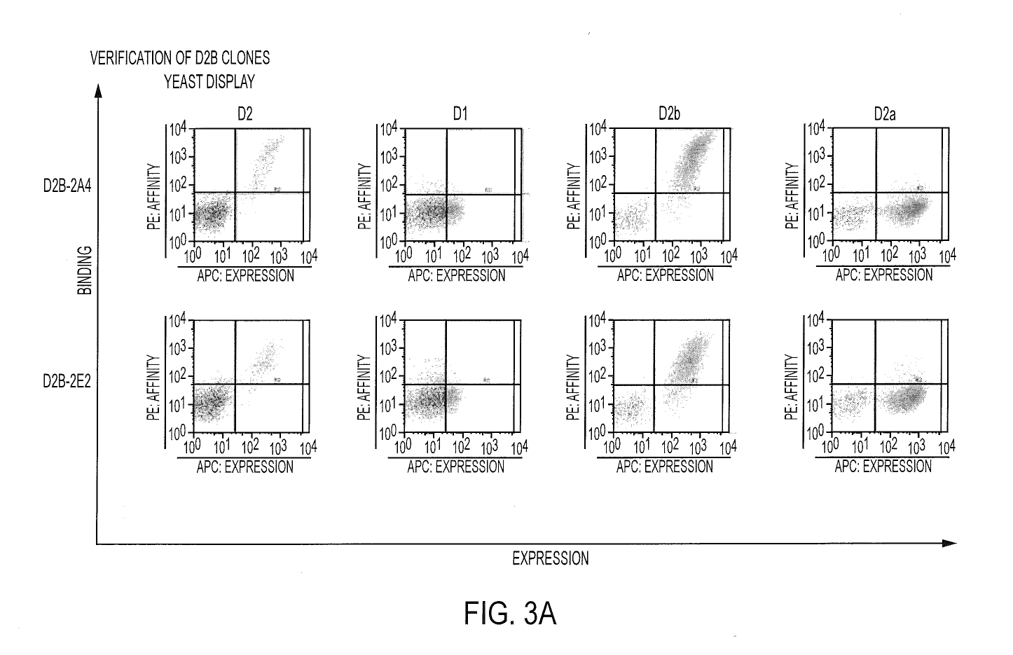

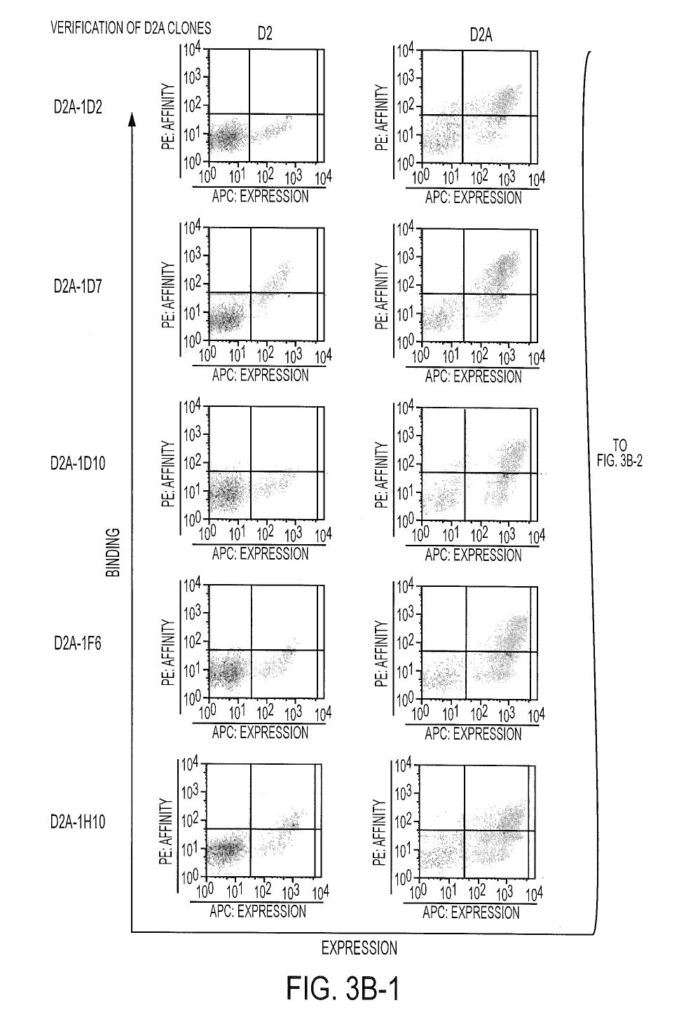

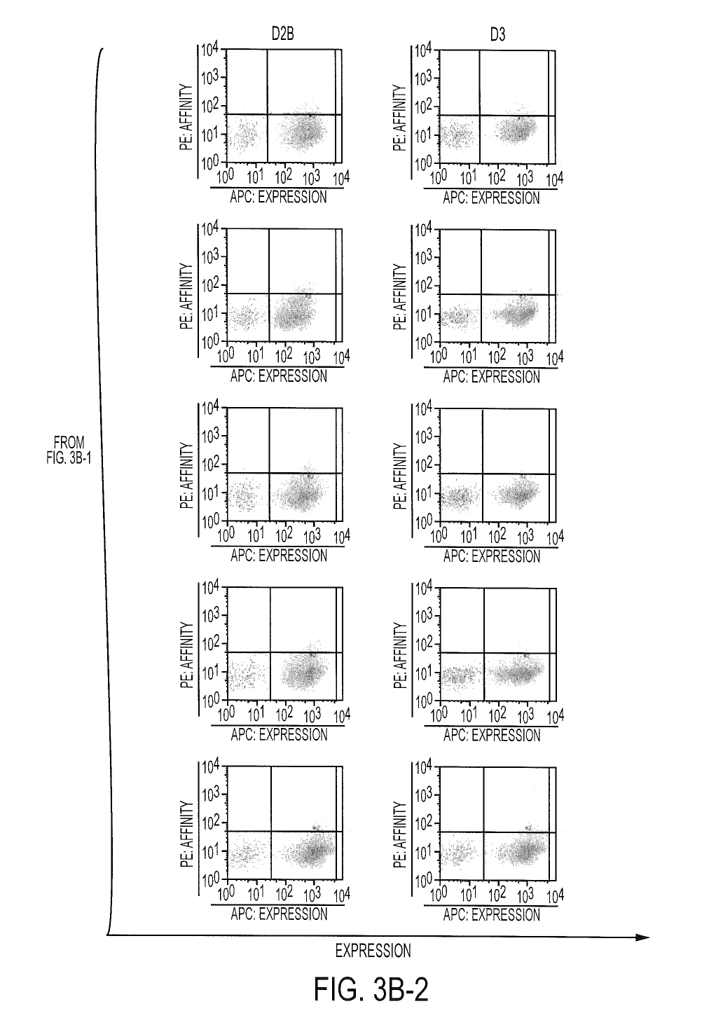

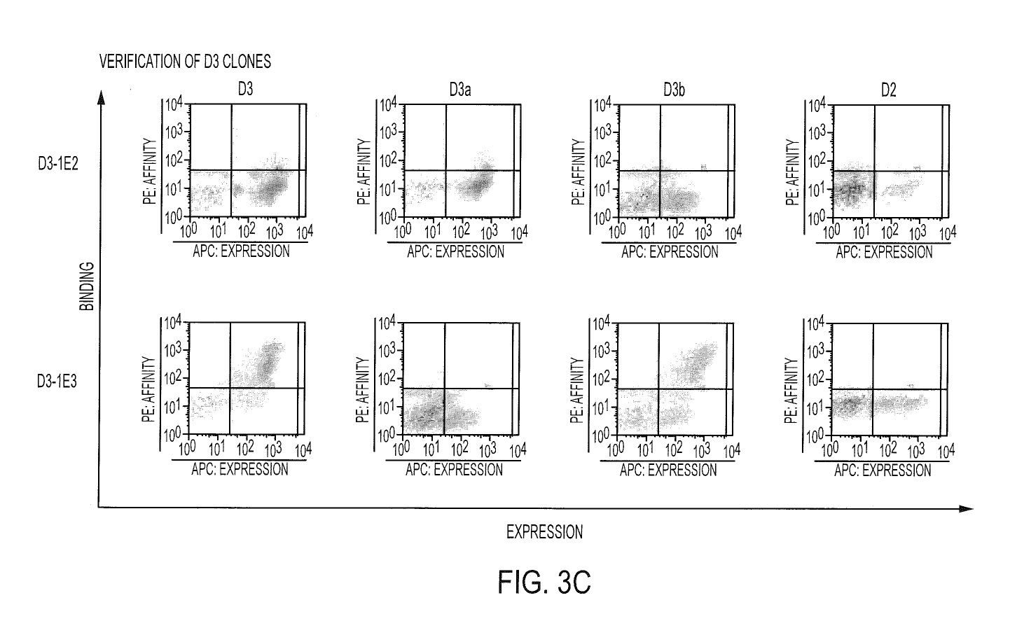

### DETAILED DESCRIPTION OF THE INVENTION

The inventors have developed human scFvs that specifically bind to extracellular domains of CSPG4 and may be internalized by cells. Internalization permits the use of the scFvs as delivery devices to tumors that express CSPG4 and as delivery devices for a payload to the interior of the targeted cells. Thus toxins and other agents that have intracellular targets can be delivered to the cytoplasm.

The human scFvs may bind to any CSPG4 extracellular domain, including amino acids 30-640; amino acids 641-1233; amino acids 1234-1586; and amino acids 1587-2222. Although not required, we utilized antibodies from non-immunized host. Immunized hosts can also be used as a starting material. Moreover, once isolated and identified, scFvs may be subjected to an affinity improvement process. We used a random mutagenesis DNA library that was expressed on yeast cell surfaces. It was initially panned against H350 melanoma cell line expressing CSPG4 on its surface. Later increasingly stringent flow cytometry sorting was used. However, any scheme can be used which selects for variants with increased affinity. Affinity may be improved by a fold of at least 2, 5, or 10.

The initial scFvs that were isolated are shown in SEQ ID NO: 1-9. The affinity matured scFvs are shown in SEQ ID NO: 10-23. These can be further modified for ease of production or increased affinity or stability, for example, without departing from the spirit of the invention.

The antibody fragments are useful for delivering agents to any cancer cells which express CSPG4 on their surfaces. These include melanoma, triple-negative breast cancer, glioblastoma, mesothelioma, osteosarcoma, clear cell renal carcinoma, head and neck squamous cell carcinoma, and sarcoma.

Any toxin or toxic agent that may kill a cell can be attached, whether post-translationally or translationally as a fusion with the scFv molecules. Suitable toxins and toxic agents include but are not limited to Diphtheria toxin, Pseudomonas aeruginosa exotoxin A shigella toxin, derivatives of these toxins, camptothecin, paclitaxel, and Vinca alkaloids,

The antibody fragments of the invention may also be useful for delivering detectable agents to tumor cells. This may facilitate monitoring of therapy or disease progression. It may facilitate early diagnosis. Detectable agents include radiolabeled molecules, fluorescent molecules, dye molecules, and the like.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

To conquer the hurdle of tumor-targeting and internalizing antibodies which are capable of delivering recombinant toxins to the cytoplasm of cancer cells effectively and specifically, we established a platform using phage display and yeast display techniques to develop human single chain variable antibody fragments (scFvs) against tumor antigens. First, different domains of the extracellular region of Chondroitin Sulfate Proteoglycan 4 (CSPG4) were displayed on the yeast surface and verified by CSPG4 mouse mAbs 9.2.27 and Mel-14. Then we constructed a human non-immunized scFv phage library, and used this library to select scFvs reactive against the different CSPG4 domains displayed on yeast surface. After multiple rounds of selection, several phage scFvs reactive against different domains of CSPG4 were chosen and analyzed by fluorescence-activated cell sorting (FACS). Finally, phage scFvs showed specific binding to melanoma cancer cell lines H350 and Malme3M.

Note: Y: yeast display; R: round; scFv, single chain variable fragments

TABLE 1
Yeast displayed domains of the extracellular region of
Chondroitin Sulfate Proteoglycan 4 (CSPG4)

| Domain Name | Amino Acid Region on CSPG4 |
|---|---|
| Domain 1 | 30-640 |
| Domain 2 | 641-1586 |
| Domain 3 | 1587-2222 |
| LG domain 1 | 30-176 |
| LG domain 2 | 203-370 |
| LG domain 1-2 | 30-370 |
| D1E | 371-640 |
| D2A | 641-1233 |
| D2B | 1234-1586 |
| D3A | 1587-1832 |
| D3B | 1833-2222 |

TABLE 2
Construction of non-immunized human scFv phage display library

| | | Insert |
|---|---|---|
| Name | Size | Percentage |
| VL library A | 4.25 × 10⁷ | 89% |
| VL library B | 2.8 × 10⁷ | 89% |
| VH library | 1.65 × 10⁸ | 84% |
| scFv library (L) | 4.0 × 10⁹ | 67% |
| scFv library (R) | 2.0 × 10⁹ | 67% |

TABLE 3
Selection of non-immunized human scFv phage display library
against yeast displayed CSPG4 domains

| | Amino Acid | | | | |
|---|---|---|---|---|---|
| Yeast- | Region of | | | | |
| Displayed | Antigen on | | | | Output:Input |
| Antigen | CSPG4 | Round | Input | Output | Ratio |
| D1 | 30-640 | 1 | 2.00e13 | 1.45e8 | 7.25e−6 |
| | | 2 | 1.00e12 | 1.68e6 | 1.68e−6 |
| | | 3 | 1.00e11 | 4.30e6 | 4.30e−5 |
| D2A | 641-1233 | 1 | 2.00e13 | 4.75e7 | 2.38e−6 |
| | | 2 | 1.00e12 | 9.34e5 | 9.34e−7 |
| | | 3 | 1.00e11 | 1.87e6 | 1.87e−5 |
| D2B | 1234-1586 | 1 | 2.00e13 | 6.23e7 | 3.12e−6 |
| | | 2 | 1.00e12 | 7.47e5 | 7.47e−7 |
| D3 | 1587-2222 | 1 | 2.00e13 | 1.04e8 | 5.20e−7 |
| | | 2 | 1.00e12 | 3.74e5 | 3.74e−7 |
| | | 3 | 1.00e11 | 9.34e5 | 9.34e−6 |

TABLE 4
Clones from outputs of selections on yeast-displayed CSPG4 domains

| Clones | Epitope | FACS (cancer cell) |
|---|---|---|
| D2A-1D2 | D2A | |
| D2A-1D7 | D2A | |
| D2A-1D10 | D2A | |
| D2A-1F6 | D2A | |
| D2A-1H10 | D2A | |
| D2B-2A4 | D2B | H350/Malme3M |
| D2B-2E2 | D2B | H350/Malme3M |
| D3-1E2 | D3B | H350/Malme3M |
| D3-1E3 | D3B | H350/Malme3M |

Cancer cell lines: H350 and Malme3M

TABLE 5
Clones from outputs of selections on yeast display CSPG4 domains

| Clones | FACS (Yeast) | FACS (cancer cell) | Epitope |
|---|---|---|---|
| D2A-1D2 | Y-D2/D2A | | D2A |
| D2A-1D7 | Y-D2/D2A | | D2A |
| D2A-1D10 | Y-D2/D2A | | D2A |
| D2A-1F6 | Y-D2/D2A | | D2A |
| D2A-1H10 | Y-D2/D2A | | D2A |
| D2B-2A4 | Y-D2/D2B | H350/Malme3M | D2B |
| D2B-2E2 | Y-D2/D2B | H350/Malme3M | D2B |
| D3B-1E2 | Y-D3/D3B | H350/Malme3M | D3B |
| D3B-1E3 | Y-D3/D3B | H350/Malme3M | D3B |

Cancer cell lines: H350 and Malme3M

### EXAMPLE 2

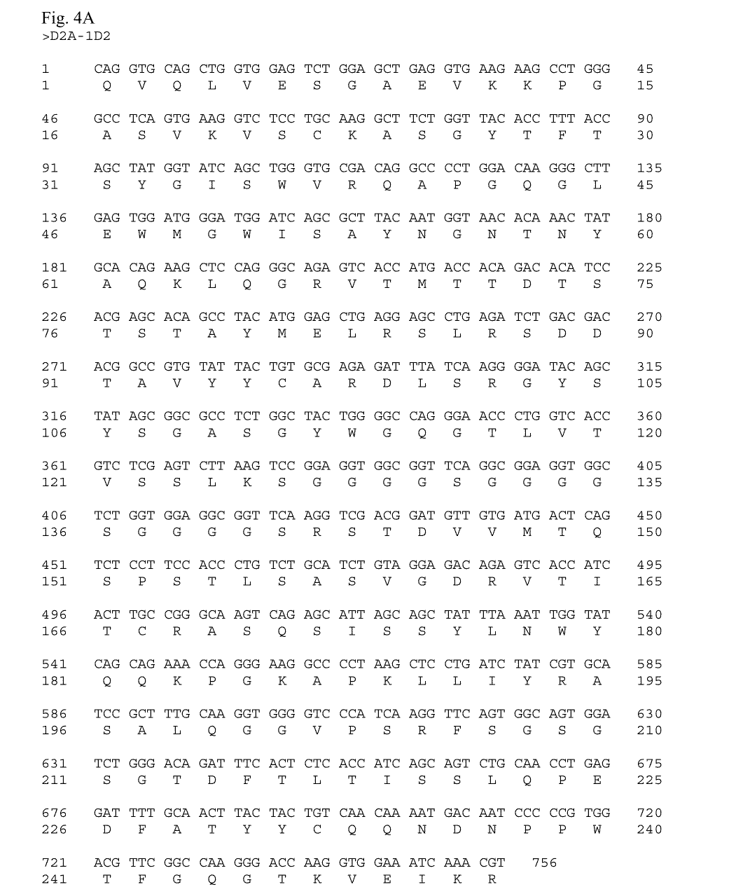

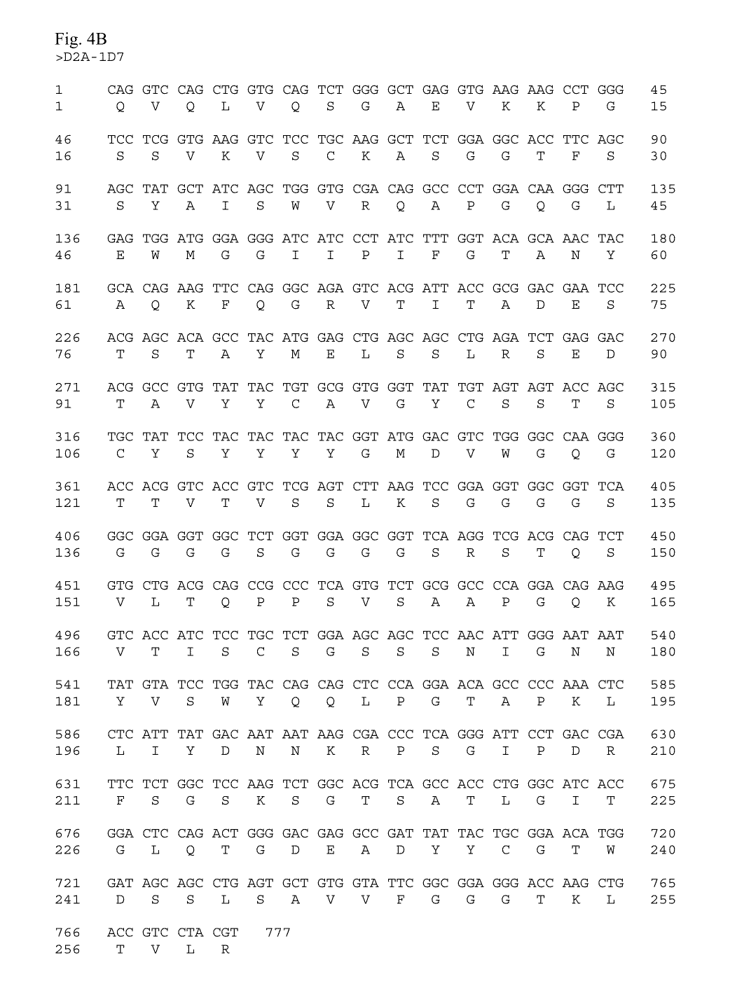

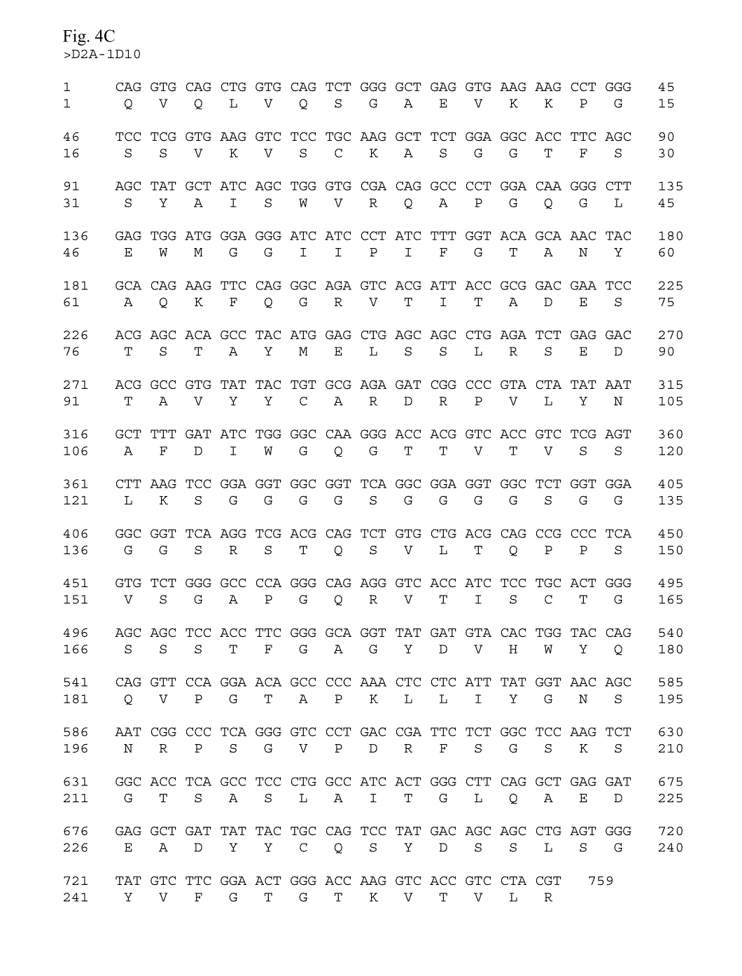

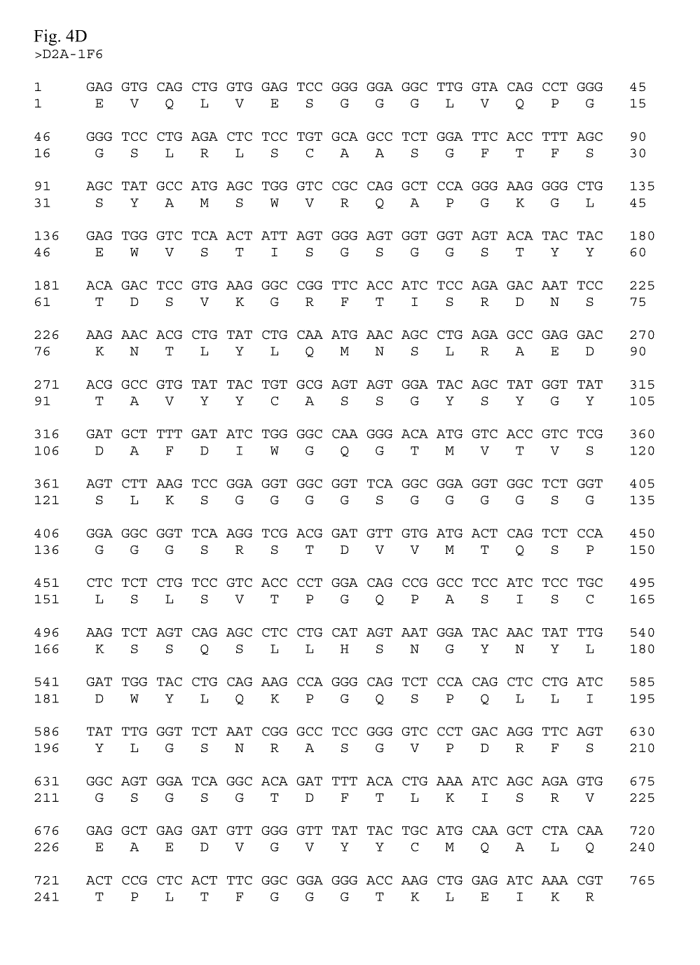

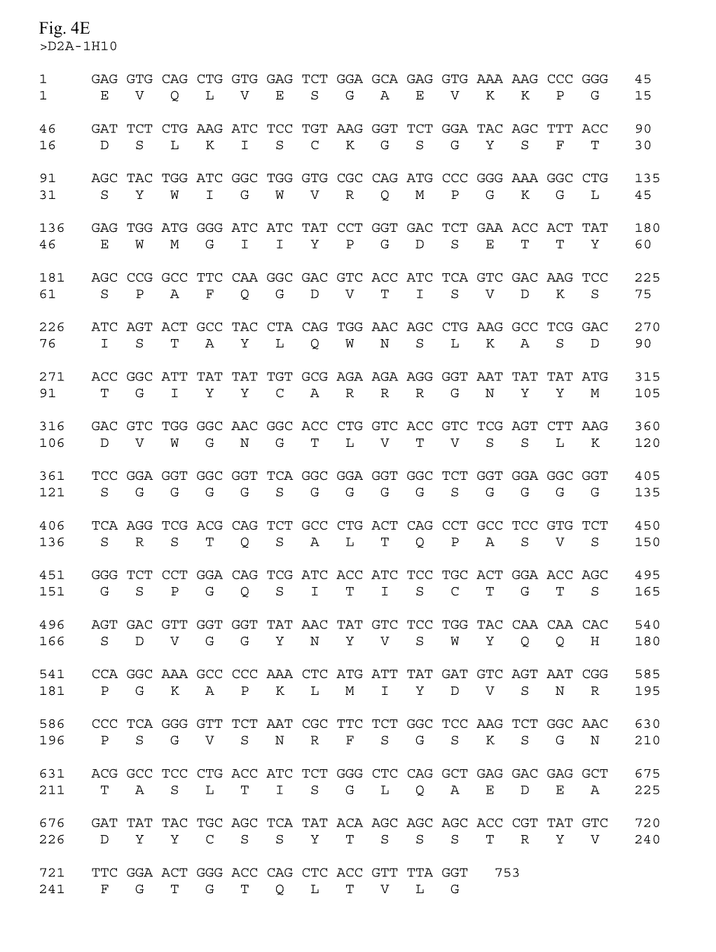

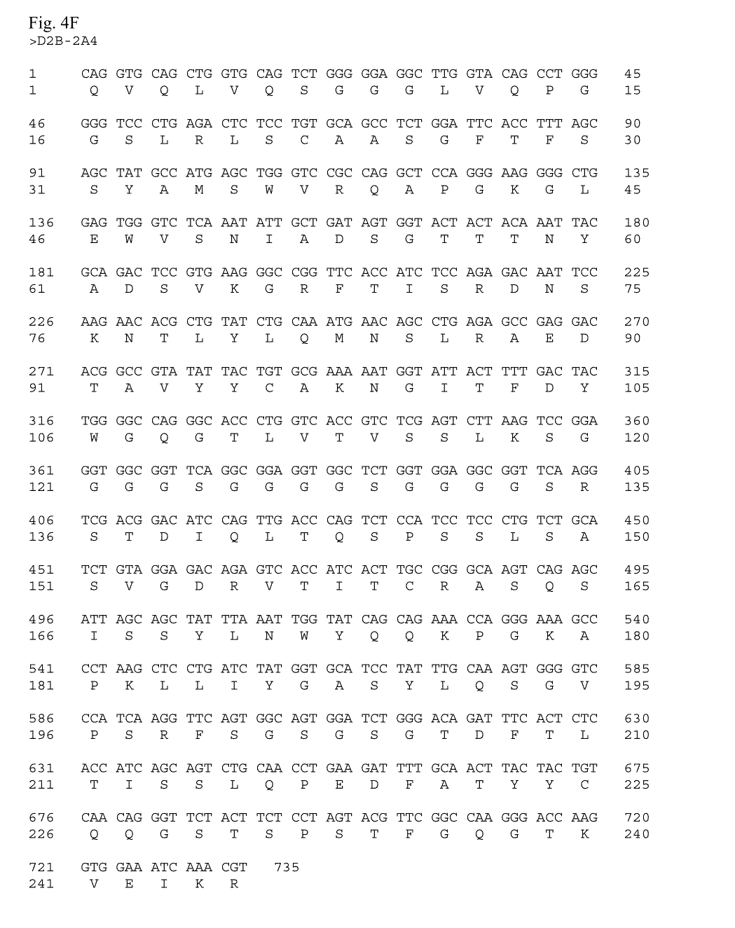

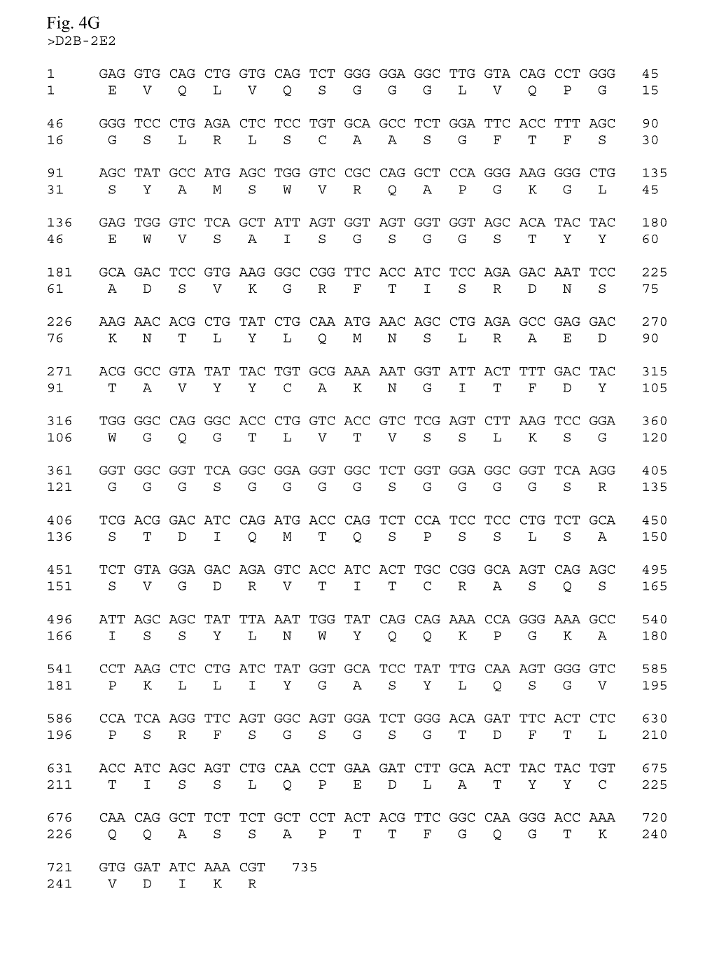

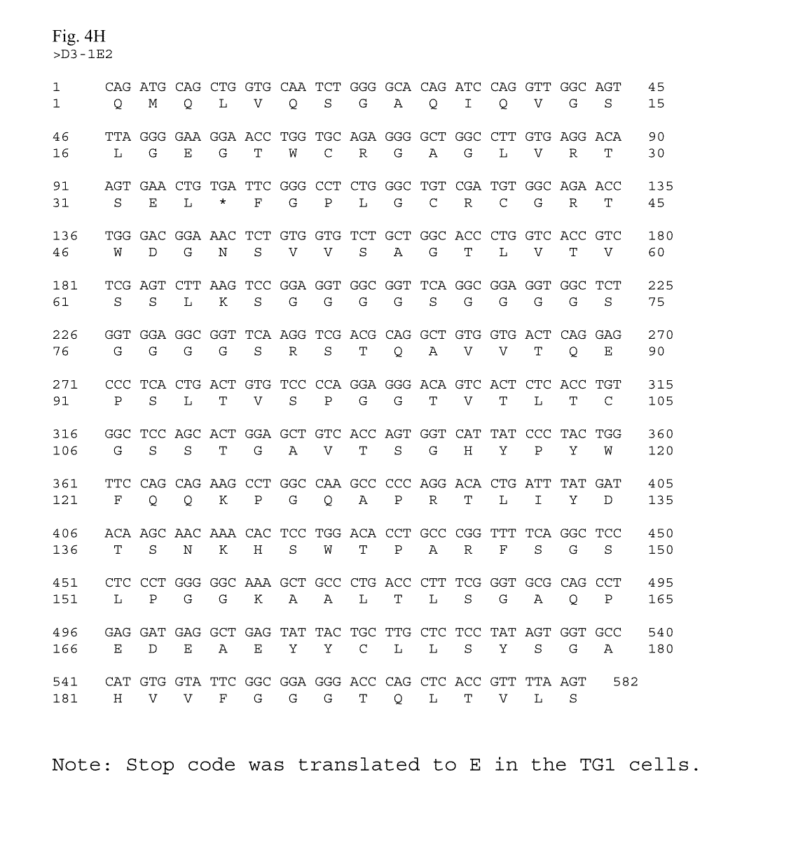

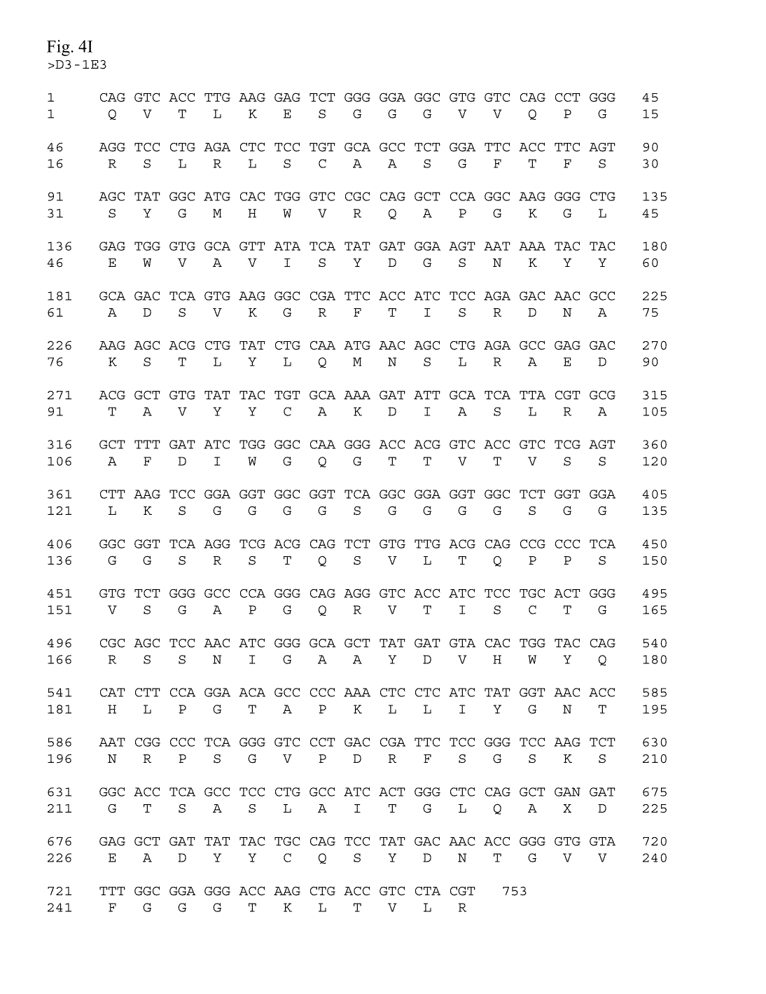

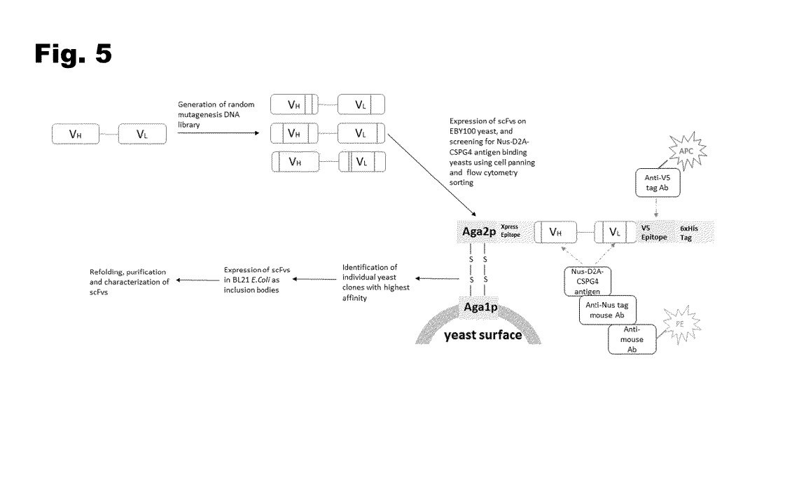

We sequenced the phage genomes which displayed useful scFv antibody fragments that we developed using this platform. The sequences are shown in FIG. 4A-4I.

### EXAMPLE 3

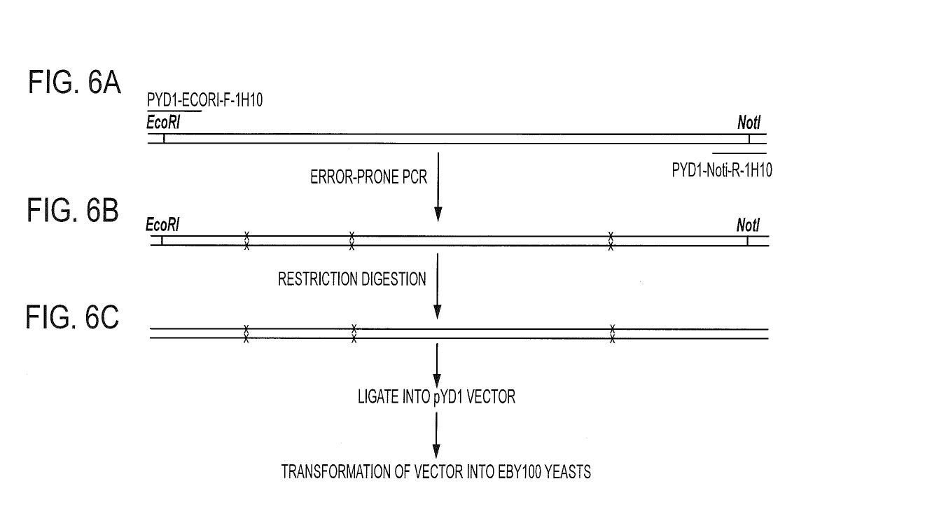

A random mutagenesis DNA library was generated based on the parental clone D2A-1H10 scFv using error-prone PCR (FIG. 6).

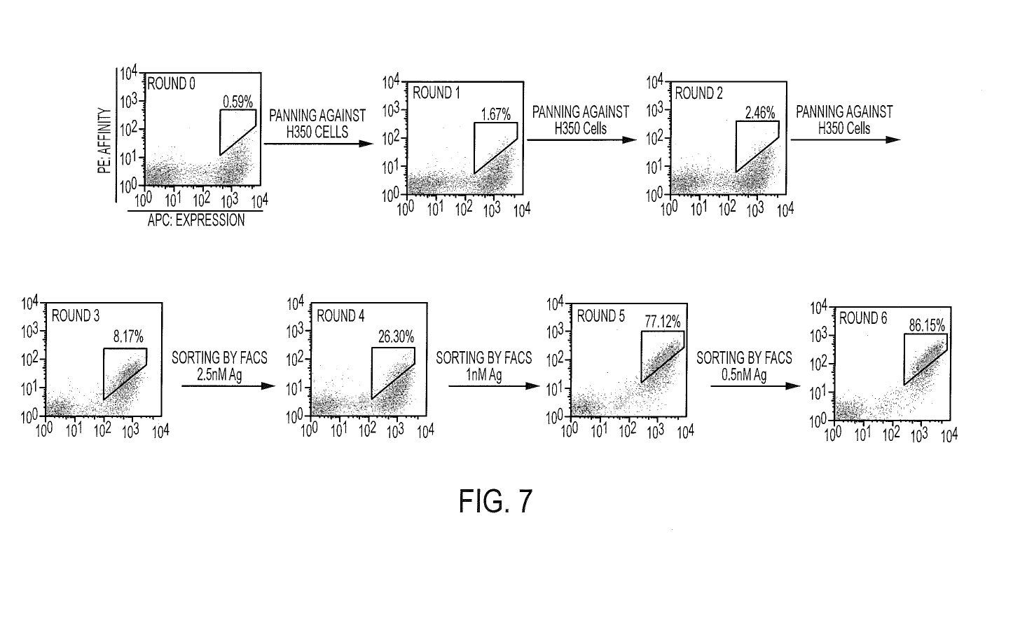

PCR generated mutant scFv DNA library was cloned into pYD1 yeast display vector and transformed into EBY100 yeast strain for screening of clones with high affinity to the D2A domain of CSPG4. The first 3 rounds of screening were performed by panning the yeast library against H350 melanoma cell line expressing CSPG4 on its surface (FIG. 7). The next 3 rounds of screening were performed by sorting the yeast clones using flow cytometry. These three rounds of flow cytometry sorting were increasingly stringent with decreased antigen concentration and narrowed sort window. As a result, significant enrichment of high affinity clones was achieved (FIG. 7).

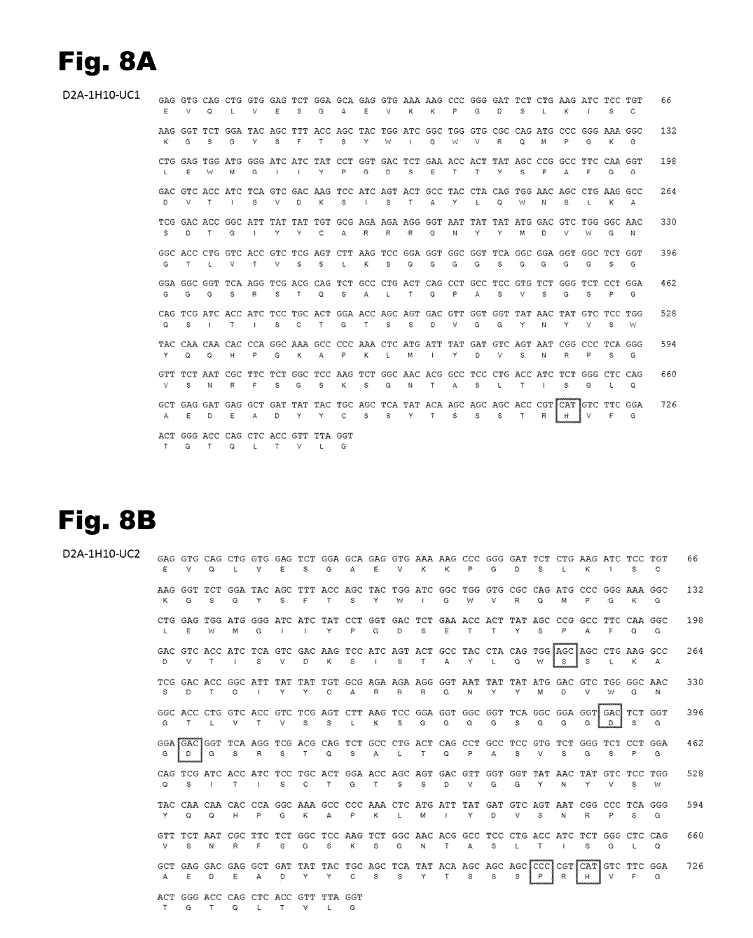

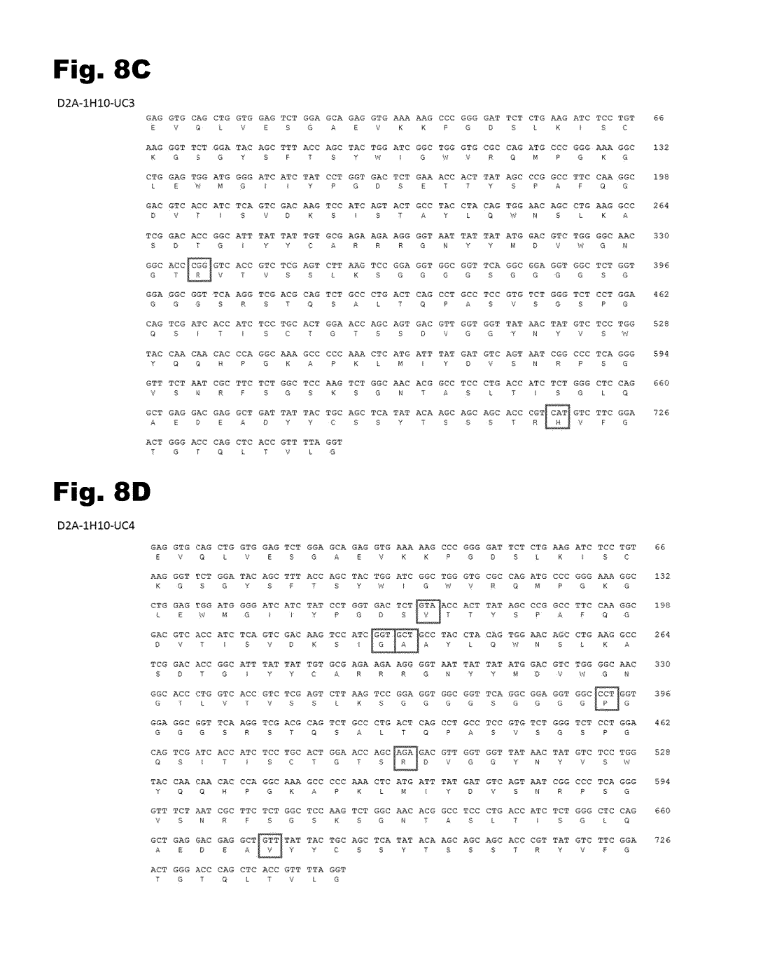

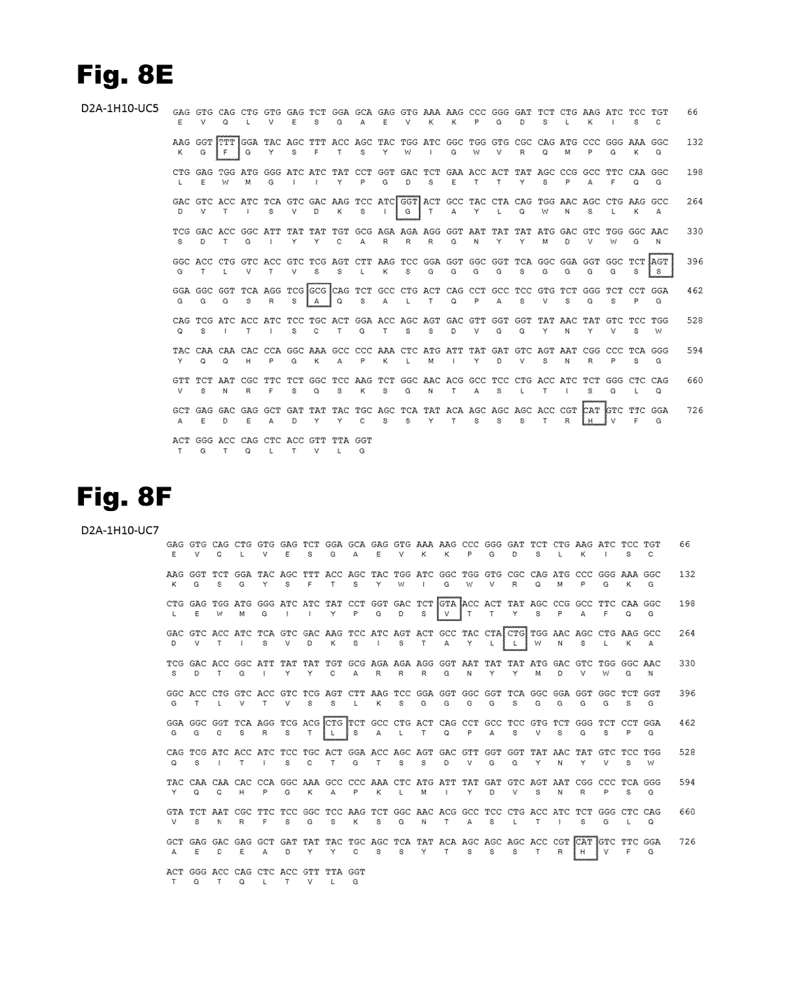

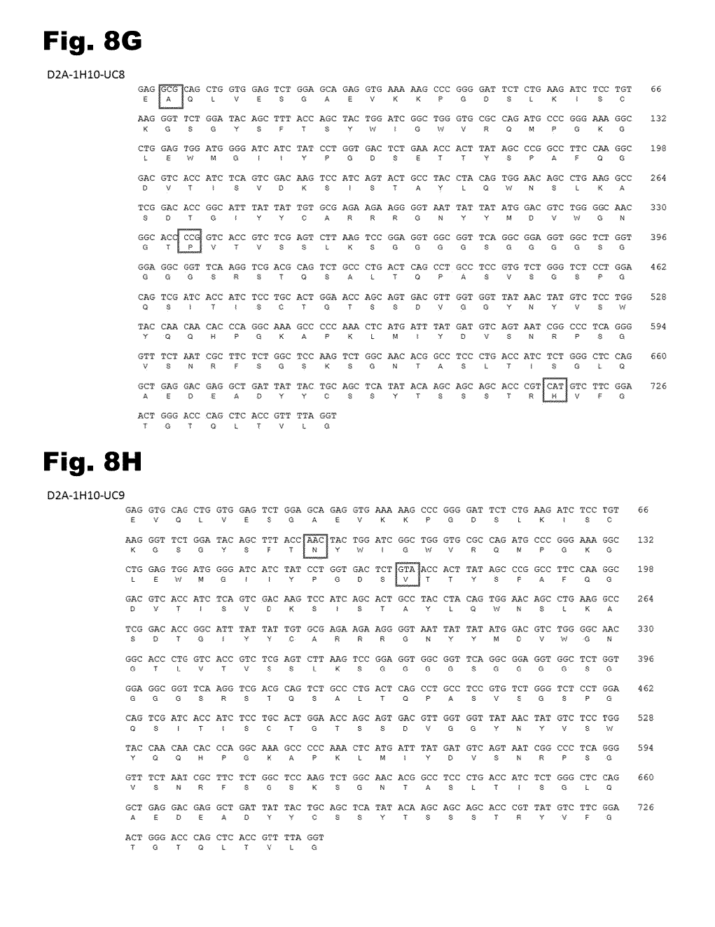

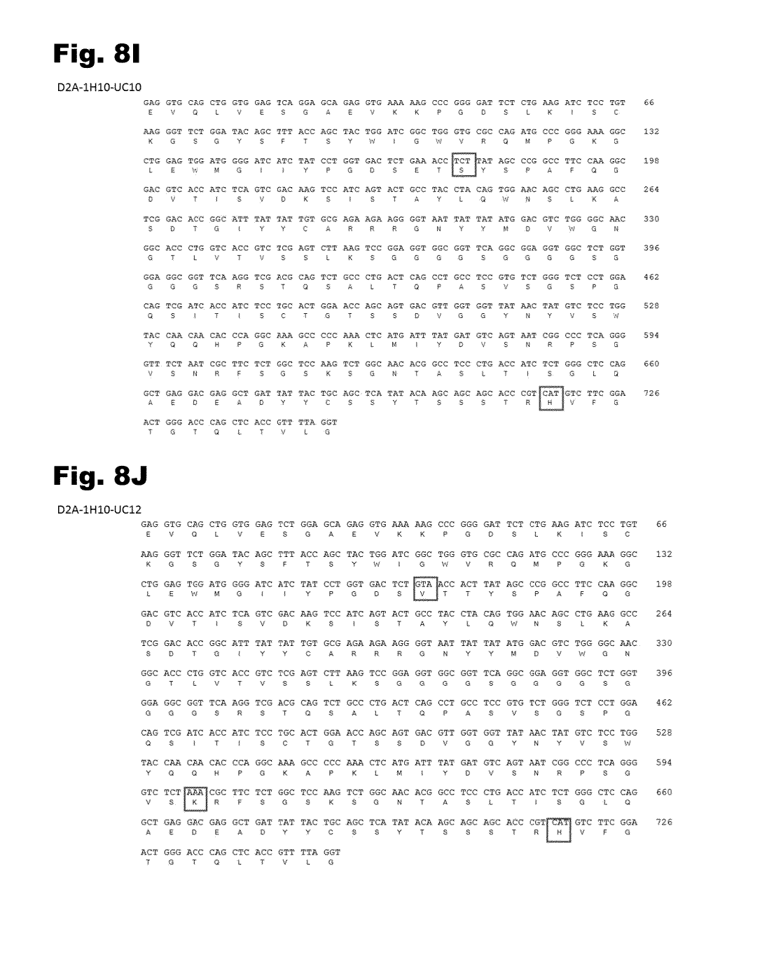

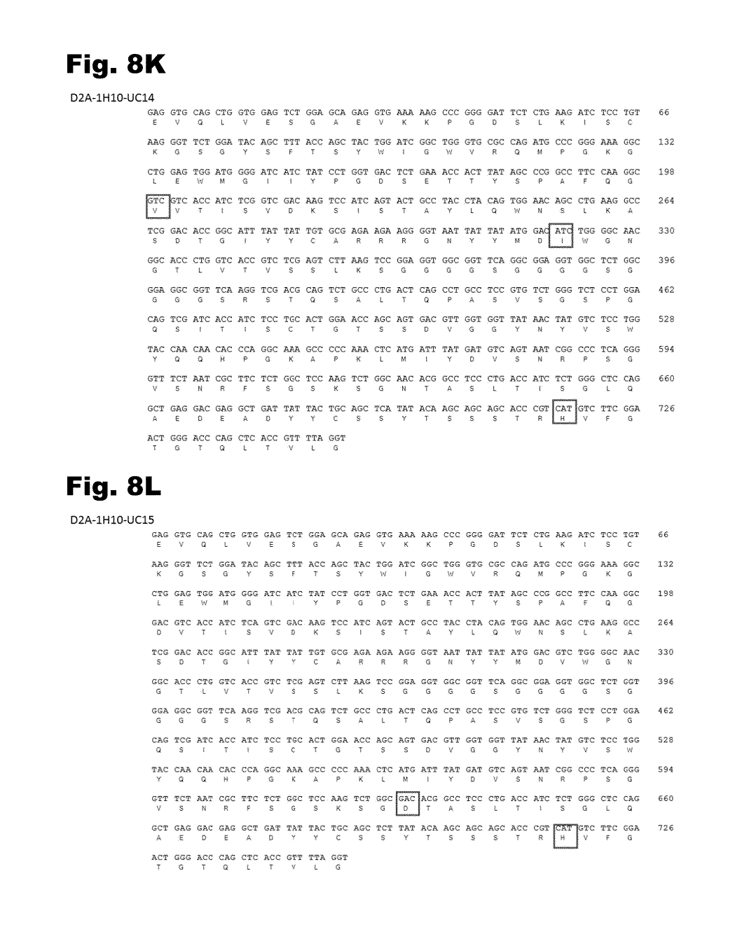

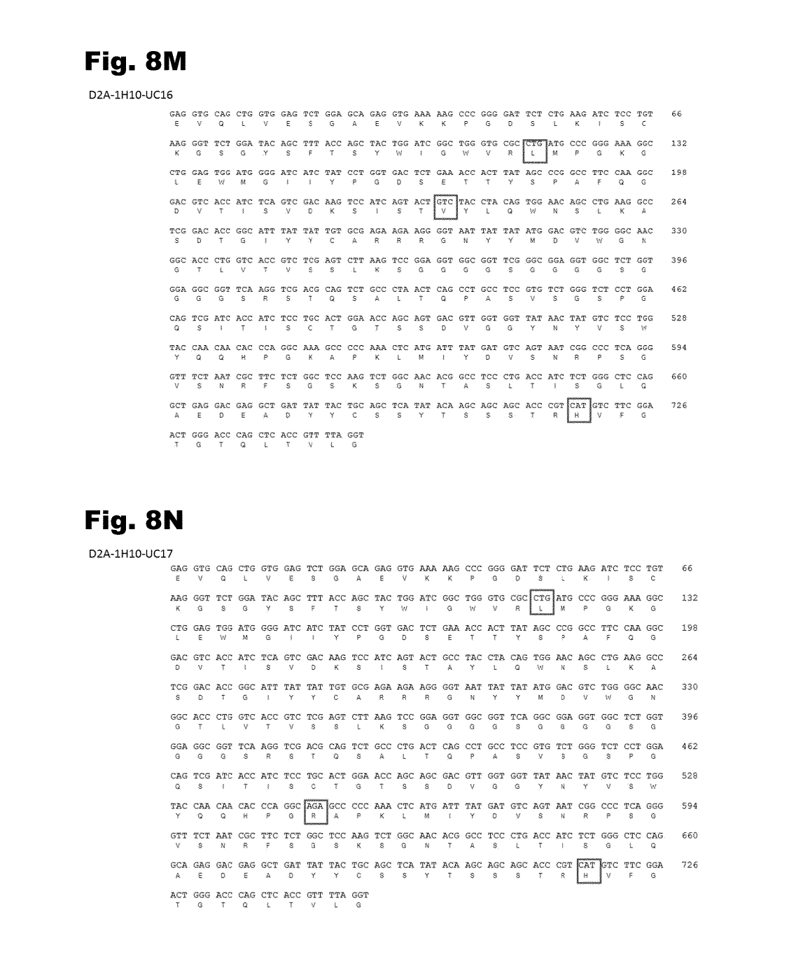

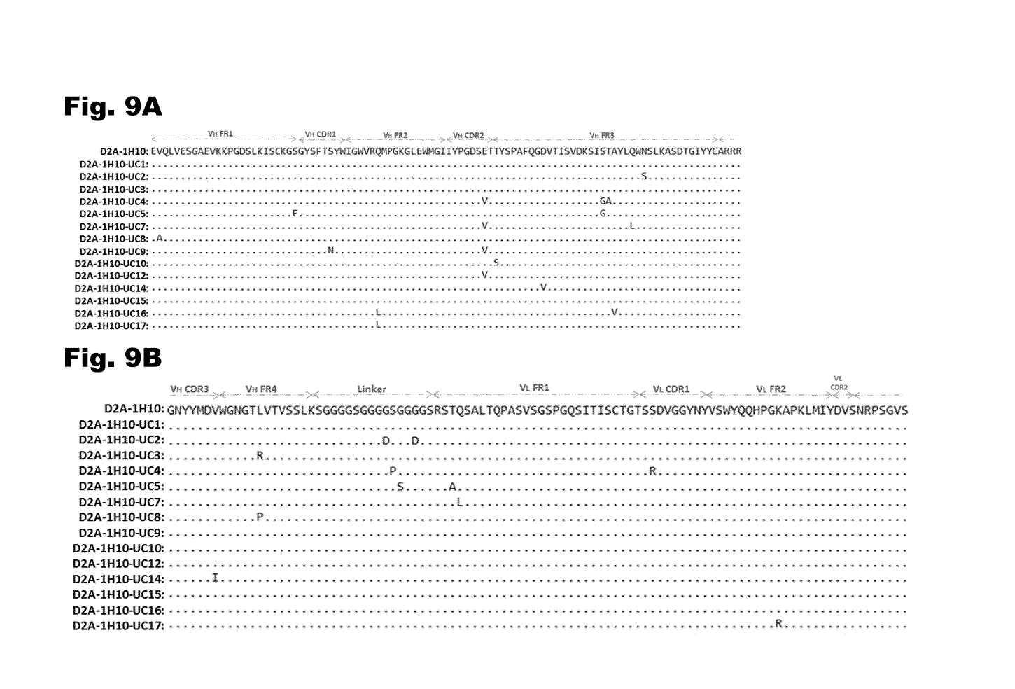

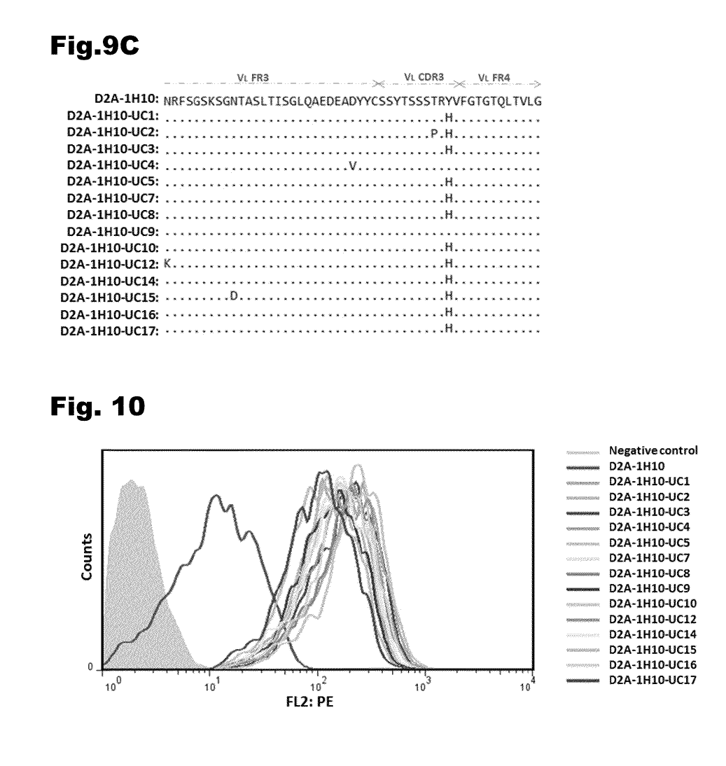

After 6 rounds of screening, 30 D2A-1H10 mutant scFv yeast clones were randomly picked up for DNA sequencing. Sequencing identified 14 unique D2A-1H10 mutant yeast clones with DNA sequence differences in the frame work and complementarity determining regions (FIG. 8). Individual D2A-1H10 mutant scFv carried approximately 2-5 amino acid mutations on average (FIG. 9). When analyzed by flow cytometry, all of the 14 unique yeast clones showed improved binding to the D2A-CSPG4 antigen compared to that of the parental clone D2A-1H10 (FIG. 10).

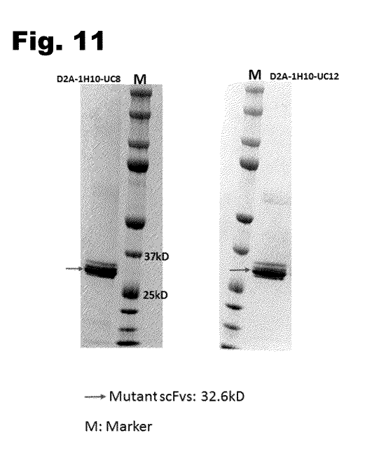

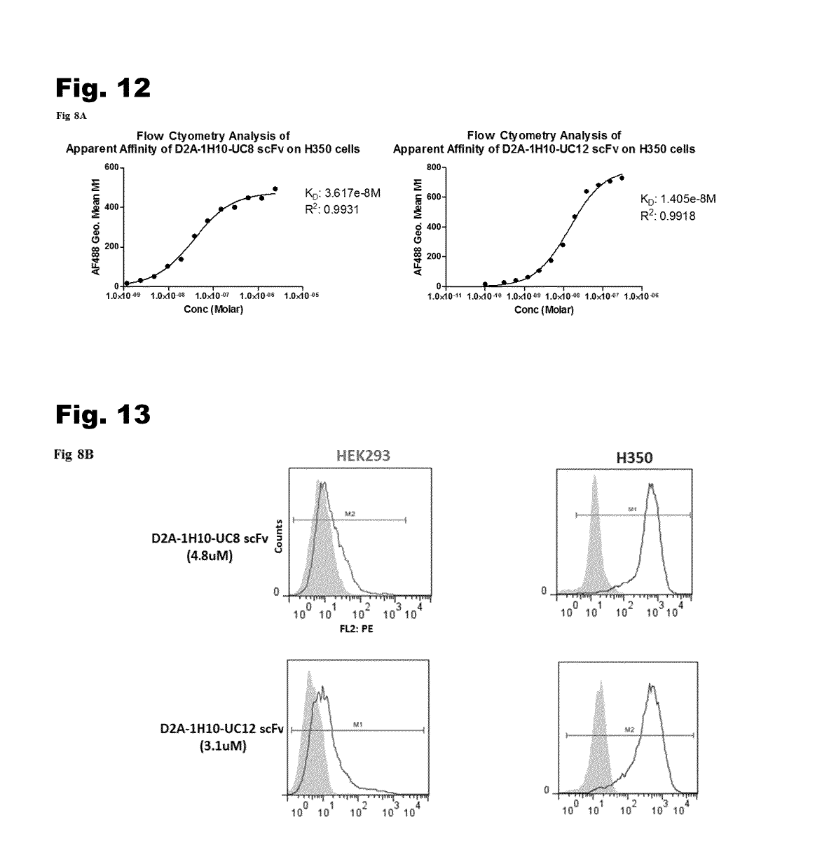

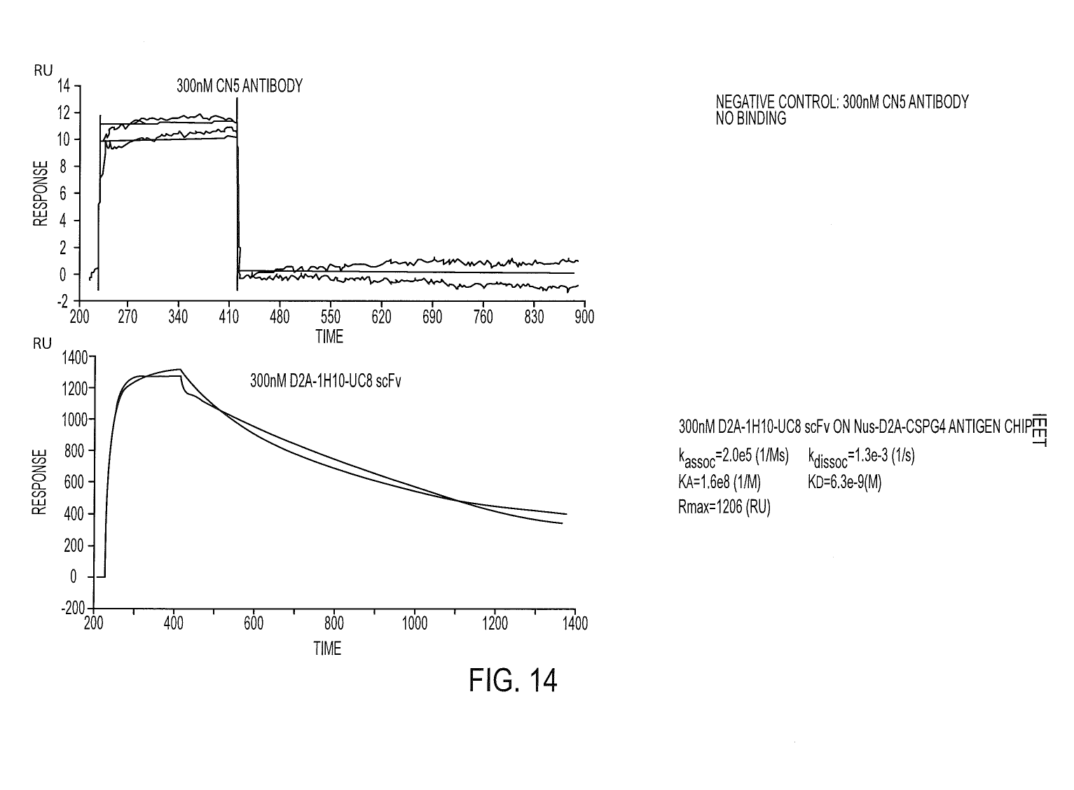

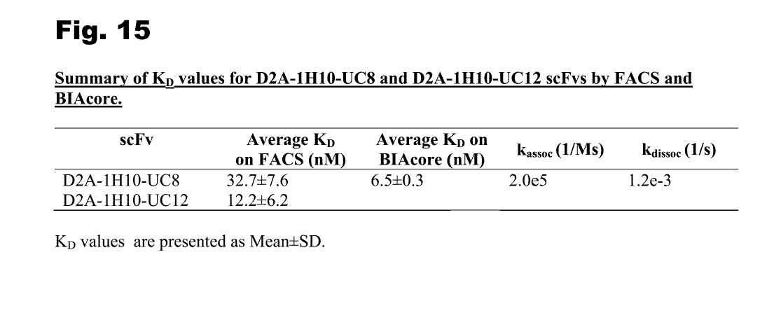

The top seven D2A-1H10 mutant scFv yeast clones demonstrating highest binding to the D2A domain of CSPG4 by FACS were selected for further characterization. Affinity (KD) of the purified D2A-1H10 mutant scFvs were determined by flow cytometry and by BIAcore (FIGS. 12-15) analysis.

We claim:

1. A human scFv that binds to an CSPG4 extracellular domain, wherein the extracellular domain is selected from the group consisting of:
a. amino acids 30-640;
b. amino acids 641-1233;
c. amino acids 1234-1586; and
d. amino acids 1587-2222.

2. The human scFv of claim 1 wherein the domain is amino acids 30-640.

3. The human scFv of claim 1 wherein the domain is 641-1233.

4. The human scFv of claim 1 wherein the domain is 1234-1586.

5. The human scFv of claim 1 wherein the domain is 1587-2222.

6. The scFv of claim 3 which has an amino acid sequence selected from the group consisting of SEQ ID NO: 1-5.

7. The scFv of claim 4 which has an amino acid sequence selected from the group consisting of SEQ ID NO: 6 and 7.

8. The scFv of claim 5 which has an amino acid sequence selected from the group consisting of SEQ ID NO: 8 and 9.

9. The scFv of claim 1 which is internalized by human cancer cells.

10. The scFv of claim 1 which is internalized by a human melanoma cells.

11. The human scFv of claim 1 which is conjugated to a toxin.

12. The human scFv of claim 1 which is fused to a toxin.

13. The human scFv of claim 3 which has the amino acid sequence of SEQ ID NO: 5 with 1-6 mutations.

14. The human scFv of claim 3 which has been affinity matured to bind to CSPG4 antigen at least 10 fold better than an scFv which as the amino acid sequence of SEQ ID NO: 5.

15. The human scFv of claim 13 which has an amino acid sequence selected from the group consisting of SEQ ID NO: 10-23.

16. The human scFv of claim 13 which has a mutation selected from the group consisting of:
a. 2A;
b. 25F;
c. 31N;
d. 39L;
e. 57V;
f. 59S;
g. 67V;
h. 77G;
i. 78A;
j. 79V;
k. 82L;
l. 84S;
m. 1071;
n. 113R;
o. 113P;
p. 130D;
q. 131P;
r. 132S;
s. 134D;
t. 139A;
u. 140L;
v. 166R;
w. 183R;
x. 201K;
y. 210D;
z. 226V;
aa. 237P; and
bb. 239H.

17. The scFv of claim 13 which comprises mutation 239H.

18. The human scFv of claim 17 which is conjugated to a toxin.

19. The human scFv of claim 18 which is fused to a toxin.


SEQUENCE LISTING (46 sequences)

SEQ ID NO: 1 - protein, 252 aa, Artificial Sequence
  single chain variable fragments
QVQLVESGAEVKKPGASVKVSCKASGYTFTSYGISWVRQAPGQGLEWMGWISAYNGNTNYAQKLQGRVTMTTDTSTSTAYMELRSLRSDDTAVYYCARDLSRGYSYSGASGYWGQGTLVTVSSLKSGGGGSGGGGSGGGGSRSTDVVMTQSPSTLSASVGDRVTITCRASQSISSYLNWYQQKPGKAPKLLIYRASALQGGVPSRFSGSGSGTDFTLTISSLQPEDFATYYCQQNDNPPWTFGQGTKVEIKR

SEQ ID NO: 2 - protein, 259 aa, Artificial Sequence
  single chain variable fragments
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAVGYCSSTSCYSYYYYGMDVWGQGTTVTVSSLKSGGGGSGGGGSGGGGSRSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVLG

SEQ ID NO: 3 - protein, 253 aa, Artificial Sequence
  single chain variable fragments
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDRPVLYNAFDIWGQGTTVTVSSLKSGGGGSGGGGSGGGGSRSTQSVLTQPPSVSGAPGQRVTISCTGSSSTFGAGYDVHWYQQVPGTAPKLLIYGNSNRPSGVPDRFSGSKSGTSASLAITGLQAEDEADYYCQSYDSSLSGYVFGTGTKVTVLR

SEQ ID NO: 4 - protein, 255 aa, Artificial Sequence
  single chain variable fragments
EVQLVESGGGLVQPGGSLRLSCAASGFTFSSYAMSWVRQAPGKGLEWVSTISGSGGSTYYTDSVKGRFTISRDNSKNTLYLQMNSLRAEDTAVYYCASSGYSYGYDAFDIWGQGTMVTVSSLKSGGGGSGGGGSGGGGSRSTDVVMTQSPLSLSVTPGQPASISCKSSQSLLHSNGYNYLDWYLQKPGQSPQLLIYLGSNRASGVPDRFSGSGSGTDFTLKISRVEAEDVGVYYCMQALQTPLTFGGGTKLEIKR

SEQ ID NO: 5 - protein, 251 aa, Artificial Sequence
  single chain variable fragments
EVQLVESGAEVKKPGDSLKISCKGSGYSFTSYWIGWVRQMPGKGLEWMGIIYPGDSETTYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRYVFGTGTQLTVLG

SEQ ID NO: 6 - protein, 245 aa, Artificial Sequence
  single chain variable fragments
QVQLVQSGGGLVQPGGSLRLSCAASGFTFSSYAMSWVRQAPGKGLEWVSNIADSGTTTNYADSVKGRFTISRDNSKNTLYLQMNSLRAEDTAVYYCAKNGITFDYWGQGTLVTVSSLKSGGGGSGGGGSGGGGSRSTDIQLTQSPSSLSASVGDRVTITCRASQSISSYLNWYQQKPGKAPKLLIYGASYLQSGVPSRFSGSGSGTDFTLTISSLQPEDFATYYCQQGSTSPSTFGQGTKVEIKR

SEQ ID NO: 7 - protein, 245 aa, Artificial Sequence
  single chain variable fragments
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKNGITFDYWGQGTLVTVSSLKSGGGGSGGGGSGGGGSRSTDIQMTQSPSSLSASVGDRVTITCRASQSISSYLNWYQQKPGKAPKLLIYGASYLQSGVPSRFSGSGSGTDFTLTISSLQPEDLATYYCQQASSAPTTFGQGTKVDIKR

SEQ ID NO: 8 - protein, 193 aa, Artificial Sequence
  single chain variable fragments
QMQLVQSGAQIQVGSLGEGTWCRGAGLVRTSELFGPLGCRCGRTWDGNSVVSAGTLVTVSSLKSGGGGSGGGGSGGGGSRSTQAVVTQEPSLTVSPGGTVTLTCGSSTGAVTSGHYPYWFQQKPGQAPRTLIYDTSNKHSWTPARFSGSLPGGKAALTLSGAQPEDEAEYYCLLSYSGAHVVFGGGTQLTVLS

SEQ ID NO: 9 - protein, 251 aa, Artificial Sequence
  single chain variable fragments
QVTLKESGGGVVQPGRSLRLSCAASGFTFSSYGMHWVRQAPGKGLEWVAVISYDGSNKYYADSVKGRFTISRDNAKSTLYLQMNSLRAEDTAVYYCAKDIASLRAAFDIWGQGTTVTVSSLKSGGGGSGGGGSGGGGSRSTQSVLTQPPSVSGAPGQRVTISCTGRSSNIGAAYDVHWYQHLPGTAPKLLIYGNTNRPSGVPDRFSGSKSGTSASLAITGLQAXDEADYYCQSYDNTGVVFGGGTKLTVLR

SEQ ID NO: 10 - protein, 251 aa, Artificial Sequence
  single chain variable fragments
EVQLVESGAEVKKPGDSLKISCKGSGYSFTSYWIGWVRQMPGKGLEWMGIIYPGDSETTYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLG

SEQ ID NO: 11 - protein, 251 aa, Artificial Sequence
  single chain variable fragments
EVQLVESGAEVKKPGDSLKISCKGSGYSFTSYWIGWVRQMPGKGLEWMGIIYPGDSETTYSPAFQGDVTISVDKSISTAYLQWSSLKASDTGIYYCARRRGNYYMDVWGNGTLVTVSSLKSGGGGSGGGDSGGDGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSPRHVFGTGTQLTVLG

SEQ ID NO: 12 - protein, 251 aa, Artificial Sequence
  single chain variable fragments
EVQLVESGAEVKKPGDSLKISCKGSGYSFTSYWIGWVRQMPGKGLEWMGIIYPGDSETTYSPAFQGDVTISVDKSISTAYLQWNSLKASDTGIYYCARRRGNYYMDVWGNGTRVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLG

SEQ ID NO: 13 - protein, 251 aa, Artificial Sequence
  single chain variable fragments
EVQLVESGAEVKKPGDSLKISCKGSGYSFTSYWIGWVRQMPGKGLEWMGIIYPGDSVTTYSPAFQGDVTISVDKSIGAAYLQWNSLKASDTGIYYCARRRGNYYMDVWGNGTLVTVSSLKSGGGGSGGGGPGGGGSRSTQSALTQPASVSGSPGQSITISCTGTSRDVGGYNYVSWYQQHPGKAPKLMIYDVSNRPSGVSNRFSGSKSGNTASLTISGLQAEDEAVYYCSSYTSSSTRYVFGTGTQLTVLG

SEQ ID NO: 14 - protein, 251 aa, Artificial Sequence
  single chain variable fragments
EVQLVESGAEVKKPGDSLKISCKGFGYSFTSYWIGWVRQMPGKGLEWMGIIYPGDSETTYSPAFQGDVTISVDKSIGTAYLQWNSLKASDTGIYYCARRRGNYYMDVWGNGTLVTVSSLKSGGGGSGGGGSSGGGSRSAQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLG

SEQ ID NO: 15 - protein, 251 aa, Artificial Sequence
  single chain variable fragments
EVQLVESGAEVKKPGDSLKISCKGSGYSFTSYWIGWVRQMPGKGLEWMGIIYPGDSVTTYSPAFQGDVTISVDKSISTAYLLWNSLKASDTGIYYCARRRGNYYMDVWGNGTLVTVSSLKSGGGGSGGGGSGGGGSRSTLSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLG

SEQ ID NO: 16 - protein, 251 aa, Artificial Sequence
  single chain variable fragments
EAQLVESGAEVKKPGDSLKISCKGSGYSFTSYWIGWVRQMPGKGLEWMGIIYPGDSETTYSPAFQGDVTISVDKSISTAYLQWNSLKASDTGIYYCARRRGNYYMDVWGNGTPVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLG

SEQ ID NO: 17 - protein, 251 aa, Artificial Sequence
  single chain variable fragments
EVQLVESGAEVKKPGDSLKISCKGSGYSFTNYWIGWVRQMPGKGLEWMGIIYPGDSVTTYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRYVFGTGTQLTVLG

SEQ ID NO: 18 - protein, 251 aa, Artificial Sequence
  single chain variable fragments
EVQLVESGAEVKKPGDSLKISCKGSGYSFTSYWIGWVRQMPGKGLEWMGIIYPGDSETSYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLG

SEQ ID NO: 19 - protein, 251 aa, Artificial Sequence
  single chain variable fragments
EVQLVESGAEVKKPGDSLKISCKGSGYSFTSYWIGWVRQMPGKGLEWMGIIYPGDSVTTYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSKRFSGSKSGNTASLTISGLQAEDEADYYCSSYTSSSTRHVFGTGTQLTVLG

SEQ ID NO: 20 - protein, 251 aa, Artificial Sequence
  single chain variable fragments
EVQLVESGAEVKKPGDSLKISCKGSGYSFTSYWIGWVRQMPGKGLEWMGIIYPGDSETTYSPAFQGVVTISVDKSISTAYLQWNSLKASDTGIYYCARRRGNYYMDIWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLG

SEQ ID NO: 21 - protein, 251 aa, Artificial Sequence
  single chain variable fragments
EVQLVESGAEVKKPGDSLKISCKGSGYSFTSYWIGWVRQMPGKGLEWMGIIYPGDSETTYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGDTASLTISGLQAEDEADYYCSSYTSSSTRHVFGTGTQLTVLG

SEQ ID NO: 22 - protein, 251 aa, Artificial Sequence
  single chain variable fragments
EVQLVESGAEVKKPGDSLKISCKGSGYSFTSYWIGWVRLMPGKGLEWMGIIYPGDSETTYSPAFQGDVTISVDKSISTVYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRHVFGTGTQLTVLG

SEQ ID NO: 23 - protein, 251 aa, Artificial Sequence
  single chain variable fragments
EVQLVESGAEVKKPGDSLKISCKGSGYSFTSYWIGWVRLMPGKGLEWMGIIYPGDSETTYSPAFQGDVTISVDKSISTAYLQWNSLKASDTGIYYCARRRGNYYMDVWGNGTLVTVSSLKSGGGGSGGGGSGGGGSRSTQSALTQPASVSGSPGQSITISCTGTSSDVGGYNYVSWYQQHPGRAPKLMIYDVSNRPSGVSNRFSGSKSGNTASLTISGLQAEDEADYYCSSYTSSSTRHVFGTGTQLTVLG

SEQ ID NO: 24 - DNA, 756 bp, Artificial Sequence
  single chain variable fragments
caggtgcagctggtggagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatttatcaaggggatacagctatagcggcgcctctggctactggggccagggaaccctggtcaccgtctcgagtcttaagtccggaggtggcggttcaggcggaggtggctctggtggaggcggttcaaggtcgacggatgttgtgatgactcagtctccttccaccctgtctgcatctgtaggagacagagtcaccatcacttgccgggcaagtcagagcattagcagctatttaaattggtatcagcagaaaccagggaaggcccctaagctcctgatctatcgtgcatccgctttgcaaggtggggtcccatcaaggttcagtggcagtggatctgggacagatttcactctcaccatcagcagtctgcaacctgaggattttgcaacttactactgtcaacaaaatgacaatcccccgtggacgttcggccaagggaccaaggtggaaatcaaacgt

SEQ ID NO: 25 - DNA, 777 bp, Artificial Sequence
  single chain variable fragments
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcggtgggttattgtagtagtaccagctgctattcctactactactacggtatggacgtctggggccaagggaccacggtcaccgtctcgagtcttaagtccggaggtggcggttcaggcggaggtggctctggtggaggcggttcaaggtcgacgcagtctgtgctgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctgtggtattcggcggagggaccaagctgaccgtcctaggt

SEQ ID NO: 26 - DNA, 759 bp, Artificial Sequence
  single chain variable fragments
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagatcggcccgtactatataatgcttttgatatctggggccaagggaccacggtcaccgtctcgagtcttaagtccggaggtggcggttcaggcggaggtggctctggtggaggcggttcaaggtcgacgcagtctgtgctgacgcagccgccctcagtgtctggggccccagggcagagggtcaccatctcctgcactgggagcagctccaccttcggggcaggttatgatgtacactggtaccagcaggttccaggaacagcccccaaactcctcatttatggtaacagcaatcggccctcaggggtccctgaccgattctctggctccaagtctggcacctcagcctccctggccatcactgggcttcaggctgaggatgaggctgattattactgccagtcctatgacagcagcctgagtgggtatgtcttcggaactgggaccaaggtcaccgtcctacgt

SEQ ID NO: 27 - DNA, 765 bp, Artificial Sequence
  single chain variable fragments
gaggtgcagctggtggagtccgggggaggcttggtacagcctggggggtccctgagactctcctgtgcagcctctggattcacctttagcagctatgccatgagctgggtccgccaggctccagggaaggggctggagtgggtctcaactattagtgggagtggtggtagtacatactacacagactccgtgaagggccggttcaccatctccagagacaattccaagaacacgctgtatctgcaaatgaacagcctgagagccgaggacacggccgtgtattactgtgcgagtagtggatacagctatggttatgatgcttttgatatctggggccaagggacaatggtcaccgtctcgagtcttaagtccggaggtggcggttcaggcggaggtggctctggtggaggcggttcaaggtcgacggatgttgtgatgactcagtctccactctctctgtccgtcacccctggacagccggcctccatctcctgcaagtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaagctggagatcaaacgt

SEQ ID NO: 28 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgaaaccacttatagcccggccttccaaggcgacgtcaccatctcagtcgacaagtccatcagtactgcctacctacagtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggctctggtggaggcggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccgttatgtcttcggaactgggacccagctcaccgttttaggt

SEQ ID NO: 29 - DNA, 735 bp, Artificial Sequence
  single chain variable fragments
caggtgcagctggtgcagtctgggggaggcttggtacagcctggggggtccctgagactctcctgtgcagcctctggattcacctttagcagctatgccatgagctgggtccgccaggctccagggaaggggctggagtgggtctcaaatattgctgatagtggtactactacaaattacgcagactccgtgaagggccggttcaccatctccagagacaattccaagaacacgctgtatctgcaaatgaacagcctgagagccgaggacacggccgtatattactgtgcgaaaaatggtattacttttgactactggggccagggcaccctggtcaccgtctcgagtcttaagtccggaggtggcggttcaggcggaggtggctctggtggaggcggttcaaggtcgacggacatccagttgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctatttgcaaagtggggtcccatcaaggttcagtggcagtggatctgggacagatttcactctcaccatcagcagtctgcaacctgaagattttgcaacttactactgtcaacagggttctacttctcctagtacgttcggccaagggaccaaggtggaaatcaaacgt

SEQ ID NO: 30 - DNA, 735 bp, Artificial Sequence
  single chain variable fragments
gaggtgcagctggtgcagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaaatggtattacttttgactactggggccagggcaccctggtcaccgtctcgagtcttaagtccggaggtggcggttcaggcggaggtggctctggtggaggcggttcaaggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctatttgcaaagtggggtcccatcaaggttcagtggcagtggatctgggacagatttcactctcaccatcagcagtctgcaacctgaagatcttgcaacttactactgtcaacaggcttcttctgctcctactacgttcggccaagggaccaaagtggatatcaaacgt

SEQ ID NO: 31 - DNA, 582 bp, Artificial Sequence
  single chain variable fragments
cagatgcagctggtgcaatctggggcacagatccaggttggcagtttaggggaaggaacctggtgcagaggggctggccttgtgaggacaagtgaactgtgattcgggcctctgggctgtcgatgtggcagaacctgggacggaaactctgtggtgtctgctggcaccctggtcaccgtctcgagtcttaagtccggaggtggcggttcaggcggaggtggctctggtggaggcggttcaaggtcgacgcaggctgtggtgactcaggagccctcactgactgtgtccccaggagggacagtcactctcacctgtggctccagcactggagctgtcaccagtggtcattatccctactggttccagcagaagcctggccaagcccccaggacactgatttatgatacaagcaacaaacactcctggacacctgcccggttttcaggctccctccctgggggcaaagctgccctgaccctttcgggtgcgcagcctgaggatgaggctgagtattactgcttgctctcctatagtggtgcccatgtggtattcggcggagggacccagctcaccgttttaagt

SEQ ID NO: 32 - DNA, 753 bp, Artificial Sequence
  single chain variable fragments
caggtcaccttgaaggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactacgcagactcagtgaagggccgattcaccatctccagagacaacgccaagagcacgctgtatctgcaaatgaacagcctgagagccgaggacacggctgtgtattactgtgcaaaagatattgcatcattacgtgcggcttttgatatctggggccaagggaccacggtcaccgtctcgagtcttaagtccggaggtggcggttcaggcggaggtggctctggtggaggcggttcaaggtcgacgcagtctgtgttgacgcagccgccctcagtgtctggggccccagggcagagggtcaccatctcctgcactgggcgcagctccaacatcggggcagcttatgatgtacactggtaccagcatcttccaggaacagcccccaaactcctcatctatggtaacaccaatcggccctcaggggtccctgaccgattctccgggtccaagtctggcacctcagcctccctggccatcactgggctccaggctgangatgaggctgattattactgccagtcctatgacaacaccggggtggtatttggcggagggaccaagctgaccgtcctacgt

SEQ ID NO: 33 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgaaaccacttatagcccggccttccaaggtgacgtcaccatctcagtcgacaagtccatcagtactgcctacctacagtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggctctggtggaggcggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggatgaggctgattattactgcagctcatatacaagcagcagcacccgtcatgtcttcggaactgggacccagctcaccgttttaggt

SEQ ID NO: 34 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgaaaccacttatagcccggccttccaaggcgacgtcaccatctcagtcgacaagtccatcagtactgcctacctacagtggagcagcctgaaggcctcggacaccggcatttattattgtgcgagaagaaggggtaattattatatggacgtctggggcaacggcaccctggtcaccgtctcgagtcttaagtccggaggtggcggttcaggcggaggtgactctggtggagacggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcccccgtcatgtcttcggaactgggacccagctcaccgttttaggt

SEQ ID NO: 35 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgaaaccacttatagcccggccttccaaggcgacgtcaccatctcagtcgacaagtccatcagtactgcctacctacagtggaacagcctgaaggcctcggacaccggcatttattattgtgcgagaagaaggggtaattattatatggacgtctggggcaacggcacccgggtcaccgtctcgagtcttaagtccggaggtggcggttcaggcggaggtggctctggtggaggcggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccgtcatgtcttcggaactgggacccagctcaccgttttaggt

SEQ ID NO: 36 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgtaaccacttatagcccggccttccaaggcgacgtcaccatctcagtcgacaagtccatcggtgctgcctacctacagtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggccctggtggaggcggttcaaggtcgacgcagtctgccctgactcagcctgcctccgtgtctgggtctcctggacagtcgatcaccatctcctgcactggaaccagcagagacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgtttattactgcagctcatatacaagcagcagcacccgttatgtcttcggaactgggacccagctcaccgttttaggt

SEQ ID NO: 37 - DNA, 753 bp, Artificial Sequence
  single chain variable fragments
gaggtgcagctggtggagtctggagcagaggtgaaaaagcccggggattctctgaagatctcctgtaagggttttggatacagctttaccagctactggatcggctgggtgcgccagatgcccgggaaaggcctggagtggatggggatcatctatcctggtgactctgaaaccacttatagcccggccttccaaggcgacgtcaccatctcagtcgacaagtccatcggtactgcctacctacagtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggctctagtggaggcggttcaaggtcggcgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccgtcatgtcttcggaactgggacccagctcaccgttttaggt

SEQ ID NO: 38 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgtaaccacttatagcccggccttccaaggcgacgtcaccatctcagtcgacaagtccatcagtactgcctacctactgtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggctctggtggaggcggttcaaggtcgacgctgtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtatctaatcgcttctccggctccaagtctggcaacacggcctccctgaccatctctgggctccaggctgaggacgaggctgattattactgcagctcatatacaagcagcagcacccgtcatgtcttcggaactgggacccagctcaccgttttaggt

SEQ ID NO: 39 - DNA, 753 bp, Artificial Sequence
  single chain variable fragments
gaggcgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgaaaccacttatagcccggccttccaaggcgacgtcaccatctcagtcgacaagtccatcagtactgcctacctacagtggaacagcctgaaggcctcggacaccggcatttattattgtgcgagaagaaggggtaattattatatggacgtctggggcaacggcaccccggtcaccgtctcgagtcttaagtccggaggtggcggttcaggcggaggtggctctggtggaggcggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccgtcatgtcttcggaactgggacccagctcaccgttttaggt

SEQ ID NO: 40 - DNA, 753 bp, Artificial Sequence
  single chain variable fragments
gaggtgcagctggtggagtctggagcagaggtgaaaaagcccggggattctctgaagatctcctgtaagggttctggatacagctttaccaactactggatcggctgggtgcgccagatgcccgggaaaggcctggagtggatggggatcatctatcctggtgactctgtaaccacttatagcccggccttccaaggcgacgtcaccatctcagtcgacaagtccatcagcactgcctacctacagtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggctctggtggaggcggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccgttatgtcttcggaactgggacccagctcaccgttttaggt

SEQ ID NO: 41 - DNA, 753 bp, Artificial Sequence
  single chain variable fragments
gaggtgcagctggtggagtcaggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgaaacctcttatagcccggccttccaaggcgacgtcaccatctcagtcgacaagtccatcagtactgcctacctacagtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggctctggtggaggcggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccgtcatgtcttcggaactgggacccagctcaccgttttaggt

SEQ ID NO: 42 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgtaaccacttatagcccggccttccaaggcgacgtcaccatctcagtcgacaagtccatcagtactgcctacctacagtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggctctggtggaggcggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtctctaaacgcttctctggctccaagtctggcaacacggcctccctgaccatctctgggctccaggctgaggacgaggctgattattactgcagctcatatacaagcagcagcacccgtcatgtcttcggaactgggacccagctcaccgttttaggt

SEQ ID NO: 43 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgaaaccacttatagcccggccttccaaggcgtcgtcaccatctcggtcgacaagtccatcagtactgcctacctacagtggaacagcctgaaggcctcggacaccggcatttattattgtgcgagaagaaggggtaattattatatggacatctggggcaacggcaccctggtcaccgtctcgagtcttaagtccggaggtggcggttcaggcggaggtggctctggcggaggcggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccgtcatgtcttcggaactgggacccagctcaccgttttaggt

SEQ ID NO: 44 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgccagatgcccgggaaaggcctggagtggatggggatcatctatcctggtgactctgaaaccacttatagcccggccttccaaggcgacgtcaccatctcagtcgacaagtccatcagtactgcctacctacagtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggctctggtggaggcggttcaaggtcgacgcagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcgacacggcctccctgaccatctctgggctccaggctgaggacgaggctgattattactgcagctcttatacaagcagcagcacccgtcatgtcttcggaactgggacccagctcaccgttttaggt

SEQ ID NO: 45 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgcctgatgcccgggaaaggcctggagtggatggggatcatctatcctggtgactctgaaaccacttatagcccggccttccaaggcgacgtcaccatctcagtcgacaagtccatcagtactgtctacctacagtggaacagcctgaaggcctcggacaccggcatttattattgtgcgagaagaaggggtaattattatatggacgtctggggcaacggcaccctggtcaccgtctcgagtcttaagtccggaggtggcggttcgggcggaggtggctctggtggaggcggttcaaggtcgacgcagtctgccctaactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccgtcatgtcttcggaactgggacccagctcaccgttttaggt

SEQ ID NO: 46 - DNA, 753 bp, Artificial Sequence
  single chain variable fragments
gaggtgcagctggtggagtctggagcagaggtgaaaaagcccggggattctctgaagatctcctgtaagggttctggatacagctttaccagctactggatcggctgggtgcgcctgatgcccgggaaaggcctggagtggatggggatcatctatcctggtgactctgaaaccacttatagcccggccttccaaggcgacgtcaccatctcagtcgacaagtccatcagtactgcctacctacagtggaacagcctgaaggcctcggacaccggcatttattattgtgcgagaagaaggggtaattattatatggacgtctggggcaacggcaccctggtcaccgtctcgagtcttaagtccggaggtggcggttcaggcggaggtggctctggtggaggcggttcaaggtcgacgcagtctgccctgactcagcctgcctccgtgtctgggtctcctggacagtcgatcaccatctcctgcactggaaccagcagcgacgttggtggttataactatgtctcctggtaccaacaacacccaggcagagcccccaaactcatgatttatgatgtcagtaatcggccctcaggggtttctaatcgcttctctggctccaagtctggcaacacggcctccctgaccatctctgggctccaggcagaggacgaggctgattattactgcagctcatatacaagcagcagcacccgtcatgtcttcggaactgggacccagctcaccgttttaggt